(19) United States
(12) Patent Application Publication
(10) Pub. No.: US 2016/0076100 A1
(43) Pub. Date: Mar. 17, 2016

(54) MSX1, DLX6 AND EDN1 AS BIOMARKERS FOR MANDIBLE SIZE

(71) Applicant: Saint Louis University, St. Louis, MO (US)

(72) Inventors: Rachel Bryn Vaughan Cooper, Houston, TX (US); Adriana M. Montano Suarez, University City, MO (US)

(21) Appl. No.: 14/855,011

(22) Filed: Sep. 15, 2015

Related U.S. Application Data
(60) Provisional application No. 62/050,298, filed on Sep. 15, 2014.

(51) Int. Cl. C12Q 1/68

(52) U.S. Cl. CPC C12Q 1/6883; C12Q 2600/158; C12Q 2600/166; C12Q 2600/124; C12Q 2600/118

(57) ABSTRACT

Disclosed are methods for diagnosing small and large mandibular sizes in individuals. Methods include determining the expression levels of MSX1, DLX6 and EDN1. Also disclosed are methods of prognosing mandibular size in an individual by determining the expression levels of MSX1, DLX6 and EDN1.

### CROSS-REFERENCE TO RELATED APPLICATIONS

This application claims the benefit under 35 U.S.C. §119(e) of U.S. Provisional Application Ser. No. 61/394,603, filed Sep. 15, 2014, the disclosure of which is incorporated herein by reference in its entirety.

### STATEMENT IN SUPPORT FOR FILING A SEQUENCE LISTING

A paper copy of the Sequence Listing and a computer readable form of the Sequence Listing containing the file named “SLU14-001_ST25.txt”, which is 29,613 bytes in size (as measured in MICROSOFT WINDOWS® EXPLORER), are provided herein and are herein incorporated by reference. This Sequence Listing consists of SEQ ID NOs:1-6.

### BACKGROUND OF THE DISCLOSURE

The present disclosure relates generally to methods for assessing mandibular growth. More particularly, the present disclosure relates to methods of diagnosing mandibular size using muscle segment homeobox 1 (MSX1), distal-less homeobox 6 (DLX6) and endothelin 1 (EDN1) expression and methods for prognosing mandibular size using MSX1, DLX6 and EDN1 expression.

The mandible forms the lower jaw (or jawbone) and holds the lower teeth in place. Mandibular size is important for providing a mechanism by which the lower face fits to the upper face. Mandibular size is also important for creating the occlusal relationship for the dentition. Disproportion in mandibular size with respect to the maxilla can result in facial distortions; affect facial aesthetics and can cause speech problems.

Many individuals start with a mandible size that is smaller than the maxilla (or the upper jaw) and rest of the face before puberty. After puberty, the mandible grows into the facial proportions. In some instances, the mandible remains disproportionately small such that the mandible remains too far behind the maxilla as determined by the position of the maxillary and mandibular incisors. Such cases can be, for example, over-bite, malocclusion or micrognathia. Although malocclusion can be common and is usually not serious enough to require treatment, severe cases of malocclusion may require orthodontic treatment, tooth extraction, growth modification and sometimes surgical treatment. A small underdeveloped mandible can also cause tooth crowding.

In other instances, the mandible can continue to grow such that the individual has an “underbite” or retrognathia in which the mandible is oversized and the maxilla is positioned posterior to the mandible as determined by the position of the maxillary and mandibular incisors. As with an undersized mandible, severe cases of an oversized mandible may require orthodontic treatment, tooth extraction, growth modification and sometimes surgical treatment.

Correction of a disproportionate mandible size may reduce the risk of tooth decay and help relieve excessive pressure on the temporomandibular joint. Correction of a disproportionate mandible size can also be done for aesthetic reasons.

Diagnosis of an undersized or oversized mandible can be determined by visual inspection as well as dental and skull X-ray. These methods depend on presentation of the undersized or oversized mandible after mandible growth is complete. Additionally, it is believed that no methods exist for prognosis of an undersized or an oversized mandible. Accordingly, there exists a need for diagnosing mandible size prior to completion of mandible growth to aid in making decisions for correction such as tooth extraction, growth modification and corrective surgery. There also exists a need for prognosis of mandible size to aid in making decisions for correction such as tooth extraction, growth modification and corrective surgery.

### SUMMARY OF THE DISCLOSURE

The present disclosure relates generally to methods for assessing mandibular growth. More particularly, the present disclosure relates to methods for diagnosing small and large mandibular size in an individual by determining the expression levels of MSX1, DLX6 and END1.

In one aspect, the present disclosure is directed to a method for diagnosing small mandibular size in an individual. The method comprises: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the target; and diagnosing small mandibular size in the individual if the expression level of MSX1 is from about 6.5 times to about 25 times less than the expression level of MSX1 from a control group; if the expression level of DLX6 is from about 5 times to about 22 times less than the expression level of DLX6 from a control group; and if the expression level of END1 is from about 1.5 times to about 10.5 times less than the expression level of EDN1 from a control group. In some embodiments, the control group includes individuals whose mandibles have a z-score calculated to be from −1 to +1 standard deviation from the average. In other embodiments, the expression level of MSX1 is from about 4.5 times to about 17.6 times less than the expression level of MSX1 from a control group, the expression level of DLX6 is from about 3.5 times to about 16.5 times less than the expression level of DLX6 from the control group, and the expression level of EDN1 is from about 1 times to about 8.2 times less than the expression level of EDN1 from the control group, wherein the control group includes individuals whose mandibles have a z-score calculated to be from −1 to 0 standard deviation from the average. In other embodiments, the expression level of MSX1 is from about 28 times to about 108 times less than the expression level of MSX1 from a control group, the expression level of DLX6 is from about 22 times to about 100 times less than the expression level of DLX6 from the control group, and the expression level of EDN1 is from about 3.5 times to about 27 times less than the expression level of EDN1 from the control group, wherein the control group includes individuals whose mandibles have a z-score calculated to be from 0 to +1 standard deviation from the average.

In another aspect, the present disclosure is directed to a method for diagnosing small mandibular size in an individual. The method comprises: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing small mandibular size in the individual if the expression level of MSX1 is from about 6.5 times to about 25 times less than the expression level of MSX1 from a control group. In some embodiments, the control group includes individuals whose mandibles have a z-score calculated to be from −1 to +1 standard deviation from the average. In other embodiments, the expression level of MSX1 is from about 4.5 times to about 17.6 times less than the expression level of MSX1 from a control group, wherein the control group includes individuals whose mandibles have a z-score calculated to be from −1 to 0 standard deviation from the average. In other embodiments, the expression level of MSX1 is from about 28 times to about 108 times less than the expression level of MSX1 from a control group, wherein the control group includes individuals whose mandibles have a z-score calculated to be from 0 to +1 standard deviation from the average.

In another aspect, the present disclosure is directed to a method for diagnosing small mandibular size in an individual. The method comprises: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing small mandibular size in the individual if the expression level of DLX6 is from about 5 times to about 22 times less than the expression level of DLX6 from the control group. In some embodiments, the control group includes individuals whose mandibles have a z-score calculated to be from −1 to +1 standard deviation from the average. In other embodiments, the expression level of DLX6 is from about 3.5 times to about 16.5 times less than the expression level of DLX6 from the control group, wherein the control group includes individuals whose mandibles have a z-score calculated to be from −1 to 0 standard deviation from the average. In other embodiments, the expression level of DLX6 is from about 22 times to about 100 times less than the expression level of DLX6 from the control group, wherein the control group includes individuals whose mandibles have a z-score calculated to be from 0 to +1 standard deviation from the average.

In another aspect, the present disclosure is directed to a method for diagnosing small mandibular size in an individual. The method comprises: obtaining a sample from the individual; contacting the sample with a probe, wherein the probe forms a complex with Endothelin 1 (EDN1); detecting the complex formed by the probe and the EDN1 to determine an expression level of EDN1; and diagnosing small mandibular size in the individual if the expression level of EDN1 is from about 1.5 times to about 10.5 times less than the expression level of EDN1 from the control group. In some embodiments, the control group includes individuals whose mandibles have a z-score calculated to be from −1 to +1 standard deviation from the average. In other embodiments, the expression level of EDN1 is from about 1 times to about 8.2 times less than the expression level of EDN1 from the control group, wherein the control group includes individuals whose mandibles have a z-score calculated to be from −1 to 0 standard deviation from the average. In other embodiments, the expression level of EDN1 is from about 3.5 times to about 27 times less than the expression level of EDN1 from the control group, wherein the control group includes individuals whose mandibles have a z-score calculated to be from 0 to +1 standard deviation from the average.

In another aspect, the present disclosure is directed to a method for diagnosing large mandibular size in an individual. The method comprises: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the nucleic acid sequence; and diagnosing large mandibular size in the individual if the expression level of MSX1 is from about 5 times to about 12 times greater than the expression level of MSX1 from a control group; if the expression level of DLX6 is from about 5.5 times to about 9.2 times greater than the expression level of DLX6 from a control group; and if the expression level of END1 is from about 1 time to about 3 times greater than the expression level of EDN1 from a control group. In some embodiments, the control group includes individuals whose mandibles have a z-score calculated to be from −1 to +1 standard deviation from the average. In other embodiments, the expression level of MSX1 is from about 3.5 times to about 8.5 times greater than the expression level of MSX1 from a control group, the expression level of DLX6 is from about 4 times to about 7 times greater than the expression level of DLX6 from a control group, and the expression level of EDN1 is from about 1 time to about 2.5 times greater than the expression level of EDN1 from the control group, wherein the control group includes individuals whose mandibles have a z-score calculated to be from −1 to 0 standard deviation from the average. In other embodiments, the expression level of MSX1 is from about 2 times to about 23 times greater than the expression level of MSX1 from a control group, the expression level of DLX6 is from about 2 times to about 41 times greater than the expression level of DLX6 from a control group, and the expression level of EDN1 is from about 2.5 time to about 8 times greater than the expression level of EDN1 from the control group, wherein the control group includes individuals whose mandibles have a z-score calculated to be from 0 to +1 standard deviation from the average.

In another aspect, the present disclosure is directed to a method for diagnosing large mandibular size in an individual. The method comprises: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing large mandibular size in the individual if the expression level of MSX1 is from about 5 times to about 12 times greater than the expression level of MSX1 from a control group. In some embodiments, the control group includes individuals whose mandibles have a z-score calculated to be from −1 to +1 standard deviation from the average. In other embodiments, the expression level of MSX1 is from about 3.5 times to about 8.5 times greater than the expression level of MSX1 from a control group, wherein the control group includes individuals whose mandibles have a z-score calculated to be from −1 to 0 standard deviation from the average. In other embodiments, the expression level of MSX1 is from about 2 times to about 23 times greater than the expression level of MSX1 from a control group, wherein the control group includes individuals whose mandibles have a z-score calculated to be from 0 to +1 standard deviation from the average.

In another aspect, the present disclosure is directed to a method for diagnosing large mandibular size in an individual. The method comprises: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing large mandibular size in the individual if the expression level of DLX6 is from about 5.5 times to about 9.2 times greater than the expression level of DLX6 from a control group. In some embodiments, the control group includes individuals whose mandibles have a z-score calculated to be from −1 to +1 standard deviation from the average. In other embodiments, the expression level of DLX6 is from about 4 times to about 7 times greater than the expression level of DLX6 from a control group, wherein the control group includes individuals whose mandibles have a z-score calculated to be from −1 to 0 standard deviation from the average. In other embodiments, the expression level of DLX6 is from about 2 times to about 41 times greater than the expression level of DLX6 from a control group, wherein the control group includes individuals whose mandibles have a z-score calculated to be from 0 to +1 standard deviation from the average.

In another aspect, the present disclosure is directed to a method for diagnosing large mandibular size in an individual. The method comprises: obtaining a sample from the individual; contacting the sample with a probe, wherein the probe forms a complex with Endothelin 1 (EDN1); detecting the complex formed by the probe and the EDN1 to determine an expression level of EDN1; and diagnosing large mandibular size in the individual if the expression level of EDN1 is from about 1 time to about 3 times greater than the expression level of EDN1 from the control group. In some embodiments, the control group includes individuals whose mandibles have a z-score calculated to be from −1 to +1 standard deviation from the average. In other embodiments, the expression level of EDN1 is from about 1 time to about 2.5 times greater than the expression level of EDN1 from the control group, wherein the control group includes individuals whose mandibles have a z-score calculated to be from −1 to 0 standard deviation from the average. In other embodiments, the expression level of EDN1 is from about 2.5 time to about 8 times greater than the expression level of EDN1 from the control group, wherein the control group includes individuals whose mandibles have a z-score calculated to be from 0 to +1 standard deviation from the average.

In another aspect, the present disclosure is directed to a method of prognosing mandibular size in an individual. The method comprises: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the target; and providing a prognosis of mandibular size, wherein the prognosis is small mandibular size in the individual if the expression level of MSX1 is from about 6.5 times to about 25 times less than the expression level of MSX1 from a control group; if the expression level of DLX6 is from about 5 times to about 22 times less than the expression level of DLX6 from a control group; and if the expression level of END1 is from about 1.5 times to about 10.5 times less than the expression level of EDN1 from a control group; and wherein the prognosis is large mandibular size in the individual if the expression level of MSX1 is from about 5 times to about 12 times greater than the expression level of MSX1 from a control group; if the expression level of DLX6 is from about 5.5 times to about 9.2 times greater than the expression level of DLX6 from a control group; and if the expression level of END1 is from about 1 time to about 3 times greater than the expression level of EDN1 from a control group.

In another aspect, the present disclosure is directed to a method of prognosing small mandibular size in an individual. The method comprises: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the target; and providing a prognosis of small mandibular size in the individual if the expression level of MSX1 is from about 6.5 times to about 25 times less than the expression level of MSX1 from a control group; if the expression level of DLX6 is from about 5 times to about 22 times less than the expression level of DLX6 from a control group; and if the expression level of END1 is from about 1.5 times to about 10.5 times less than the expression level of EDN1 from a control group. In some embodiments, the control group includes individuals whose mandibles have a z-score calculated to be from −1 to +1 standard deviation from the average. In other embodiments, the expression level of MSX1 is from about 4.5 times to about 17.6 times less than the expression level of MSX1 from a control group, the expression level of DLX6 is from about 3.5 times to about 16.5 times less than the expression level of DLX6 from the control group, and the expression level of EDN1 is from about 1 times to about 8.2 times less than the expression level of EDN1 from the control group, wherein the control group includes individuals whose mandibles have a z-score calculated to be from −1 to 0 standard deviation from the average. In other embodiments, the expression level of MSX1 is from about 28 times to about 108 times less than the expression level of MSX1 from a control group, the expression level of DLX6 is from about 22 times to about 100 times less than the expression level of DLX6 from the control group, and the expression level of EDN1 is from about 3.5 times to about 27 times less than the expression level of EDN1 from the control group, wherein the control group includes individuals whose mandibles have a z-score calculated to be from 0 to +1 standard deviation from the average.

In another aspect, the present disclosure is directed to a method of prognosing large mandibular size in an individual. The method comprises: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the nucleic acid sequence; and providing a prognosis of large mandibular size in the individual if the expression level of MSX1 is from about 5 times to about 12 times greater than the expression level of MSX1 from a control group; if the expression level of DLX6 is from about 5.5 times to about 9.2 times greater than the expression level of DLX6 from a control group; and if the expression level of END1 is from about 1 time to about 3 times greater than the expression level of EDN1 from a control group. In some embodiments, the control group includes individuals whose mandibles have a z-score calculated to be from −1 to +1 standard deviation from the average. In other embodiments, the expression level of MSX1 is from about 3.5 times to about 8.5 times greater than the expression level of MSX1 from a control group, the expression level of DLX6 is from about 4 times to about 7 times greater than the expression level of DLX6 from a control group, and the expression level of EDN1 is from about 1 time to about 2.5 times greater than the expression level of EDN1 from the control group, wherein the control group includes individuals whose mandibles have a z-score calculated to be from −1 to 0 standard deviation from the average. In other embodiments, the expression level of MSX1 is from about 2 times to about 23 times greater than the expression level of MSX1 from a control group, the expression level of DLX6 is from about 2 times to about 41 times greater than the expression level of DLX6 from a control group, and the expression level of EDN1 is from about 2.5 time to about 8 times greater than the expression level of EDN1 from the control group, wherein the control group includes individuals whose mandibles have a z-score calculated to be from 0 to +1 standard deviation from the average.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic illustration showing a mandible with the Condylion and Gnathion points of measurement and dimension line (D1) illustrating the measurement path to assess mandibular size as discussed herein.

FIG. 2 is an enlargement of the condylion of the mandible as illustrated in FIG. 1.

FIG. 3 is an enlargement of the gnathion of the mandible as illustrated in FIG. 1.

FIG. 4 is a graphical illustration of the number of subjects per group as discussed in Example 1.

FIG. 5 is a graph depicting the number of subjects analyzed for EDN1 as discussed in Example 1.

FIG. 6 a graph depicting the number of subjects analyzed for DLX6 as discussed in Example 1.

FIG. 7 a graph depicting the number of subjects analyzed for MSX1 as discussed in Example 1.

FIG. 8 a graph depicting the threshold cycle (Ct) average of gene expression as discussed in Example 1.

FIG. 9 a graph depicting the fold regulation for subjects expressing MSX1 in comparison to the average of Groups 2 and 3 as discussed in Example 1.

FIG. 10 a graph depicting the fold regulation for subjects expressing DLX6 in comparison to the average of Groups 2 and 3 as discussed in Example 1.

FIG. 11 a graph depicting the fold regulation for subjects expressing EDN1 in comparison to the average of Groups 2 and 3 as discussed in Example 1.

FIG. 12 is a graph depicting the fold regulation for subjects expressing MSX1 in comparison to the average of Group 2 as discussed in Example 1.

FIG. 13 is a graph depicting the fold regulation for subjects expressing DLX6 in comparison to the average of Group 2 as discussed in Example 1.

FIG. 14 is a graph depicting the fold regulation for subjects expressing EDN1 in comparison to the average of Group 2 as discussed in Example 1.

FIG. 15 is a graph depicting the fold regulation for subjects expressing MSX1 in comparison to the average of Group 3 as discussed in Example 1.

FIG. 16 is a graph depicting the fold regulation for subjects expressing DLX6 in comparison to the average of Group 3 as discussed in Example 1.

FIG. 17 is a graph depicting the fold regulation for subjects expressing EDN1 in comparison to the average of Group 3 as discussed in Example 1.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

In accordance with the present disclosure, methods have been developed for diagnosing small mandible size in an individual. Methods have also been developed for diagnosing large mandible size in an individual. The expression level of MSX1, DLX6 and EDN1 can be used to diagnose small mandible size in an individual and to diagnose large mandible size in an individual. Methods have also been developed for prognosing mandible size in an individual. The expression level of MSX1, DLX6 and EDN1 can be used to prognose an individual's mandible size. Diagnosing small mandible size or large mandible size in individuals and prognosing mandible size in individuals allow for treatment, treatment planning, and treatment options for mandibular undergrowth or mandibular overgrowth.

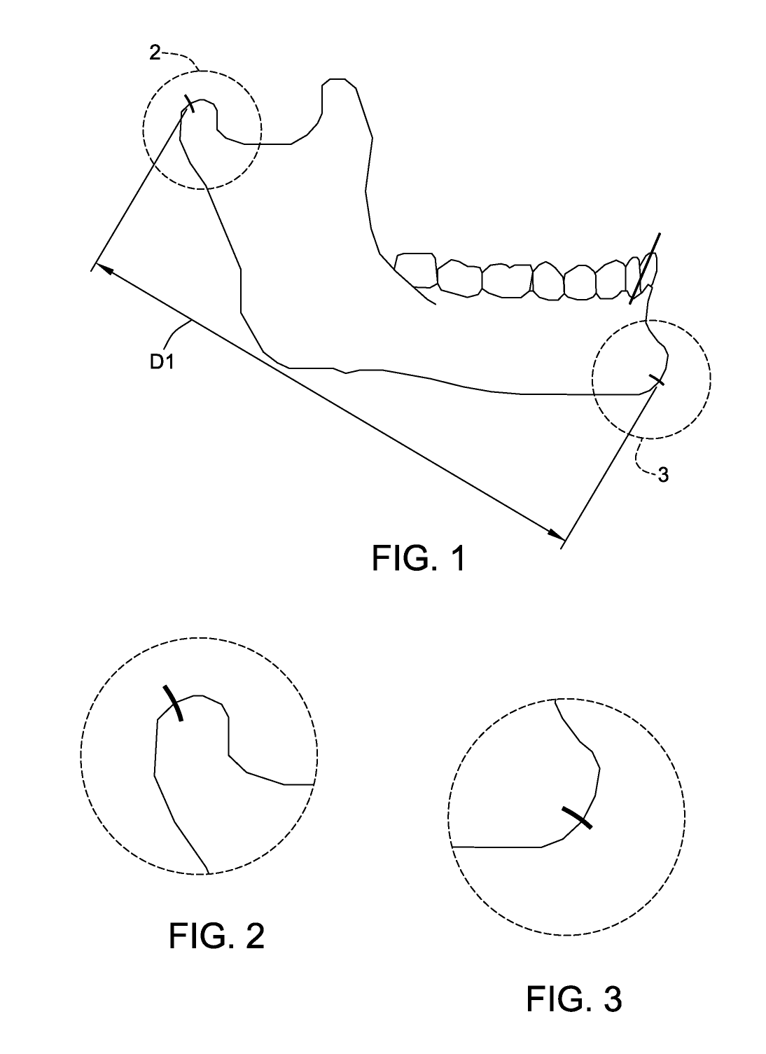

In some embodiments, the methods of the present disclosure as described herein are intended to include the use of such methods in “at risk” individuals, including individuals unaffected by or not otherwise afflicted with mandibular undergrowth (referred to herein as “small mandible size) or mandibular overgrowth (referred to herein as “large mandible size”) as described herein, for the purpose of diagnosing, prognosing and identifying individuals such that treatment, treatment planning, and treatment options for mandibular undergrowth or mandibular overgrowth can be made. As used herein, an individual “at risk for small mandible size” refers to individuals who may develop a small mandible size due to undergrowth of the individual's mandible. Additionally, an individual “at risk for large mandible size” refers to individuals who may develop a large mandible size due to overgrowth of the individual's mandible. As such, in some embodiments, the methods disclosed herein are directed to a subset of the general population such that, in these embodiments, not all of the general population may benefit from the methods. Based on the foregoing, because some of the method embodiments of the present disclosure are directed to specific subsets or subclasses of identified individuals (that is, the subset or subclass of individuals “at risk for” the specific conditions noted herein), not all individuals will fall within the subset or subclass of individuals as described herein. The terms “mandible size” and “mandibular size” are used interchangeably herein to refer to the measurement from condylion to gnathion, referred to herein as “the Condylion-Gnathion (Co-Gn) measurement”. The terms “small mandible size” and “small mandibular size” are used interchangeably herein to refer to the measurement from condylion to gnathion, wherein the Co-Gn measurement is more than one standard deviation below the average mandible measurement obtained from a control group. The terms “large mandible size” and “large mandibular size” are interchangeably used herein to refer to the measurement from condylion to gnathion, wherein the Co-Gn measurement is more than one standard deviation above the average mandible measurement obtained from a control. To determine mandible size (the “Co-Gn measurement”), measurement of an X-ray (radiograph) of the individual's mandible from condylion to gnathion is recorded. Radiographs can be obtained using cephalometric radiograph machines known to those skilled in the art (e.g., SIDEXIS, Sirona Dental Systems, Inc., Long Island City, N.Y.; i-CAT®, Imaging Sciences International, Hatfield, Pa.; KODAK, Atlanta, Ga.). As illustrated in FIG. 1 and known to those skilled in the art, the condylion is the lateral tip of the condyle (“Co”) of the human mandible (see, FIG. 2) and the gnathion (“Gn”) is the midpoint of the lower border of the human mandible (see, FIG. 3). The Co-Gn measurement is taken along dimension line (D1) as illustrated in FIG. 1 by placing the points correctly in the radiograph and the measurement is computed using computer software (e.g., QUICK CEPH® Systems, San Diego, Calif.). Due to magnification differences of each cephalometric radiograph machine, the measurements are adjusted to match the magnification obtained while taking the x-ray. To match the magnification, a ruler can be placed on the radiograph machine that will appear in the radiograph. Two dots are placed in the software to obtain the ratio between the actual ruler and the magnified image of the ruler. Then the software adjusts the measurements according to this magnification to provide the value in millimeters. A standard or z-score can then be calculated for the Co-Gn mandibular measurement. The z-score calculates in standard deviations how far the Co-Gn measurement is from the average. The individual can then be sorted into a group based on the diagnosis of Pierre Robin sequence (or Pierre Robin syndrome) and the z-score. In one embodiment, three groups can be identified based on the Pierre Robin sequence and the z-score. In another embodiment, four groups can be identified based on the Pierre Robin sequence and the z-score. Averages and standard deviations for each age group can then be determined using Moyer's standards as described herein.

In a particularly preferred embodiment, to determine whether the Co-Gn measurement is more than one standard deviation below (less than) the average mandible Co-Gn measurement obtained from a control group or more than one standard deviation above (greater than) the average mandible Co-Gn measurement obtained from a control group, the Co-Gn measurement obtained from measurement of X-rays of the individual's mandible from condylion to gnathion can be compared to the average mandible size shown in Table 1 (below). As such, a particularly preferred average mandible Co-Gn measurement from a control group for determining whether the individual's mandible Co-Gn measurement is more than one standard deviation below the average mandible Co-Gn measurement obtained from a control group or more than one standard deviation above the average mandible Co-Gn measurement obtained from a control group can be determined from Table 1 of the present disclosure. Using the values provided in Table 1, an average mandible Co-Gn measurement can also be calculated based on age, sex and combinations thereof.

As used herein, “diagnosing” and “diagnosis” are used according to their ordinary meaning as understood by those skilled in the art to refer to identifying that an individual is likely to develop or is at risk for developing a small mandibular size and/or identifying that an individual is likely to develop or is at risk for developing a large mandibular size.

As used herein, the term “target” refers to a molecule to be used for analyzing an individual's test sample. Examples of such targets can be nucleic acids (such as, for example, a gene, DNA and RNA), proteins and polypeptides. In particularly preferred embodiments, the target can be a target gene. Particularly suitable target genes can be, for example, the Muscle segment homeobox (Msh) 1 (MSX1) gene, the Distal-less homeobox 6 (DLX6) gene, the Endothelin 1 (EDN1) gene, and combinations thereof.

Muscle segment homeobox (Msh) 1 (MSX1) is a protein that in humans is encoded by the MSX1 gene (see, Accession NG 008121). MSX1 has been implicated in the development of palate, teeth and other craniofacial structures (Han et al., Devel. Biol. 2003, 261:183-196; Satokata and Maas, Nat Genet 1994; 6:348-56). In humans, mutations in the MSX1 gene result in orofacial clefting and tooth agenesis, consistent with the phenotype observed in Msx1 mutant mice (Hu et al., Mol. Cell. Biol. 1998, 18:6044-6051; Jumlongras et al., Am. J. Hum. Gen. 2001, 69(1):67-74; van den Boogaard et al., Nat Genet 2000, 24:342-343; Vastardis et al., Nat Genet 1996, 13:417-421). In mice, Msx1 is required for Bmp4 and Bmp2 expression in the palatal mesenchyme and Shh expression in the palatal epithelium. Shh acts downstream of Bmp4 and upstream of Bmp2 to stimulate mesenchymal cell proliferation to promote the outgrowth of the palatal shelf (Zhang et al., Devel. 2002, 129:4135-4146).

Distal-less homeobox 6 (DLX6) is a protein that in humans is encoded by the DLX6 gene (see, Accession NC 000007). DLX6 belongs to a homeobox transcription factor gene family similar to the Drosophila distal-less gene. The DLX family includes at least 6 different members that encode proteins with roles implicated in forebrain and craniofacial development.

Endothelin 1 (EDN1), also known as preproendothelin-1 (PPET1), is a protein that is encoded in humans by the EDN1 gene (see, Accession NG 016196). Endothelin 1 protein is proteolytically processed to release a secreted peptide that functions as a potent vasoconstrictor and is produced by vascular endothelial cells.

### Methods for Diagnosing Small Mandibular Size

In one aspect, the present disclosure is directed to a method for diagnosing small mandibular size in an individual. In one embodiment, the present disclosure is directed to a method for diagnosing an individual at risk for small mandibular size.

The method includes: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the target; and diagnosing small mandibular size in the individual if the expression level of MSX1 is from about 6.5 times to about 25 times less than the expression level of MSX1 from a control group; if the expression level of DLX6 is from about 5 times to about 22 times less than the expression level of DLX6 from a control group; and if the expression level of END1 is from about 1.5 times to about 10.5 times less than the expression level of EDN1 from a control group. In some embodiments, the control group includes individuals whose mandibles have a z-score calculated to be from −1 to +1 standard deviation from the average. In other embodiments, the expression level of MSX1 is from about 4.5 times to about 17.6 times less than the expression level of MSX1 from a control group, the expression level of DLX6 is from about 3.5 times to about 16.5 times less than the expression level of DLX6 from the control group, and the expression level of EDN1 is from about 1 times to about 8.2 times less than the expression level of EDN1 from the control group, wherein the control group includes individuals whose mandibles have a z-score calculated to be from −1 to 0 standard deviation from the average. In other embodiments, the expression level of MSX1 is from about 28 times to about 108 times less than the expression level of MSX1 from a control group, the expression level of DLX6 is from about 22 times to about 100 times less than the expression level of DLX6 from the control group, and the expression level of EDN1 is from about 3.5 times to about 27 times less than the expression level of EDN1 from the control group, wherein the control group includes individuals whose mandibles have a z-score calculated to be from 0 to +1 standard deviation from the average.

In one aspect, the present disclosure is directed to a method for diagnosing small mandibular size in an individual by measuring Muscle segment homeobox 1 (MSX1). In one embodiment, the present disclosure is directed to a method for diagnosing an individual as at risk for small mandibular size by measuring Muscle segment homeobox 1 (MSX1). The method includes: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing small mandibular size the individual if the expression level of MSX1 is from about 6.5 times to about 25 times less than the expression level of MSX1 from a control group. In this embodiment, the control group includes individuals whose mandibles have a z-score calculated to be from −1 to +1 standard deviation from the average.

In another embodiment, the present disclosure is directed to a method for diagnosing small mandibular size in an individual by measuring Muscle segment homeobox 1 (MSX1). The method includes: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing small mandibular size in the individual if the expression level of MSX1 is from about 4.5 times to about 17.6 times less than the expression level of MSX1 from a control group. In this embodiment, the control group (the “moderate small group”) includes individuals whose mandibles have a z-score calculated to be from −1 to 0 standard deviation from the average.

In another embodiment, the present disclosure is directed to a method for diagnosing small mandibular size in an individual by measuring Muscle segment homeobox 1 (MSX1). The method includes: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing small mandibular size in the individual if the expression level of MSX1 is from about 28 times to about 108 times less than the expression level of MSX1 from a control group. In this embodiment, the control group (the “moderate large group”) includes individuals whose mandibles have a z-score calculated to be from 0 to +1 standard deviation from the average.

In another embodiment, the present disclosure is directed to a method for diagnosing small mandibular size in an individual by measuring Distal-less homeobox 6 (DLX6). The method includes: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing small mandibular size in an individual if the expression level of DLX6 is from about 5 times to about 22 times less than the expression level of DLX6 from the control group. In this embodiment, the control group includes individuals whose mandibles have a z-score calculated to be from −1 to +1 standard deviation from the average.

In another embodiment, the present disclosure is directed to a method for diagnosing small mandibular size in an individual by measuring Distal-less homeobox 6 (DLX6). The method includes: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing small mandibular size in an individual if the expression level of DLX6 is from about 3.5 times to about 16.5 times less than the expression level of DLX6 from the control group. In this embodiment, the control group includes individuals whose mandibles have a z-score calculated to be from −1 to 0 standard deviation from the average.

In another embodiment, the present disclosure is directed to a method for diagnosing small mandibular size in an individual by measuring Distal-less homeobox 6 (DLX6). The method includes: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing small mandibular size in an individual if the expression level of DLX6 is from about 22 times to about 100 times less than the expression level of DLX6 from the control group. In this embodiment, the control group includes individuals whose mandibles have a z-score calculated to be from 0 to +1 standard deviation from the average.

In another embodiment, the present disclosure is directed to a method for diagnosing small mandibular size in an individual by measuring Endothelin 1 (EDN1). The method includes: obtaining a sample from the individual; contacting the sample with a probe, wherein the probe forms a complex with Endothelin 1 (EDN1); detecting the complex formed by the probe and the EDN1 to determine an expression level of EDN1; and diagnosing small mandibular size in an individual size if the expression level of EDN1 is from about 1.5 times to about 10.5 times less than the expression level of EDN1 from the control group. In this embodiment, the control group includes individuals whose mandibles have a z-score calculated to be from −1 to +1 standard deviation from the average.

In another embodiment, the present disclosure is directed to a method for diagnosing small mandibular size in an individual by measuring Endothelin 1 (EDN1). The method includes: obtaining a sample from the individual; contacting the sample with a probe, wherein the probe forms a complex with Endothelin 1 (EDN1); detecting the complex formed by the probe and the EDN1 to determine an expression level of EDN1; and diagnosing small mandibular size in an individual if the expression level of EDN1 is from about 1 times to about 8.2 times less than the expression level of EDN1 from the control group. In this embodiment, the control group includes individuals whose mandibles have a z-score calculated to be from −1 to 0 standard deviation from the average.

In another embodiment, the present disclosure is directed to a method for diagnosing small mandibular size in an individual by measuring Endothelin 1 (EDN1). The method includes: obtaining a sample from the individual; contacting the sample with a probe, wherein the probe forms a complex with Endothelin 1 (EDN1); detecting the complex formed by the probe and the EDN1 to determine an expression level of EDN1; and diagnosing small mandibular size in an individual if the expression level of EDN1 is from about 3.5 times to about 27 times less than the expression level of EDN1 from the control group. In this embodiment, the control group includes individuals whose mandibles have a z-score calculated to be from 0 to +1 standard deviation from the average.

The level of expression of MSX1, DLX6 and/or EDN1 can be analyzed using methods such as, for example, reverse transcription polymerase chain reaction (RT-PCR), Northern blot analysis, Southern blot analysis, RNase protection assays, microarrays, serial analysis of gene expression (SAGE), Western blot analysis, immunoprecipitation and combinations thereof.

Reverse transcription polymerase chain reaction (RT-PCR) of gene expression can be used to detect MSX1, DLX6 and EDN1 expression levels by creating complementary DNA from RNA obtained from a sample from an individual.

A particularly suitable RT-PCR method for detecting MSX1, DLX6 and EDN1 expression levels is quantitative PCR (RT-qPCR).

For RT-qPCR, the minimum information for publication of quantitative real-time PCR experiments (MIQE) guidelines are used to ensure the technical quality of the methodology to produce data that are consistent, comparable and reliable (see, Taylor et al., “A Practical Approach to RT-qPCR—Publishing Data That Conform to the MIQE Guidelines,” Bio-Rad Amplification Tech Note 5859 (2011); Bustin et al., “The MIQE guidelines: minimum information for publication of quantitative real-time PCR experiments,” Clin. Chem. 55:611-622 (2009), the disclosures of which are hereby incorporated by reference in their entireties). MIQE provides a standardized approach for each step of the RT-qPCR workflow to ensure reliable and reproducible results.

Northern blot analysis can be performed using methods known to those skilled in the art. For example, total RNA can be extracted from the individual's saliva sample. RNA having a poly(A) tail can then be isolated. The isolated RNA can then be separated according to size by electrophoresis and transferred to a membrane by a blotting system such as, for example, vacuum blotting or capillary blotting. The membrane can then be exposed to the probes described herein, wherein the probes form a complex with the target RNA (e.g., MSX1, DLX6 and EDN1). The complex can then be detected using known methods such as, for example, radioactivity, colorimetric and chemiluminescence.

The MSX1, DLX6 and EDN1 level can be measured by quantifying the signal by densitometry of X-ray film using a densitometer.

Suitable samples can be, for example, saliva, blood, plasma, serum and a cheek swab. The samples can be further processed using methods known to those skilled in the art to isolate molecules contained in the sample such as, for example, cells, proteins and nucleic acids (e.g., DNA and RNA).

The isolated molecules can also be further processed. For example, cells can be lysed and subjected to methods for isolating proteins and/or nucleic acids contained within the cells. Proteins and nucleic acids contained in the sample and/or in isolated cells can be processed. For example, proteins can be processed for electrophoresis, Western blot analysis, immunoprecipitation and combinations thereof. Nucleic acids can be processed, for example, for polymerase chain reaction, electrophoresis, Northern blot analysis, Southern blot analysis, RNase protection assays, microarrays, serial analysis of gene expression (SAGE) and combinations thereof.

Suitable probes are described herein and can include, for example, nucleic acid probes, antibody probes, and chemical probes. A particularly suitable probe can be, for example, an oligonucleotide probe labelled with SYBR® Green. Particularly suitable nucleic acid probes can be oligonucleotide probes designed using the MSX1 (SEQ ID NO:1), DLX6 (SEQ ID NO:3) and EDN1 (SEQ ID NO:5) nucleotide sequences provided herein. Particularly suitable antibody probes can be designed using the MSX1 (SEQ ID NO:2), DLX6 (SEQ ID NO:4) and EDN1 (SEQ ID NO:6) amino acid sequences provided herein.

In some embodiments, the probe can be a labeled probe. Suitable labels can be, for example, a fluorescent label, an enzyme label, a radioactive label, a chemical label, and combinations thereof. Suitable radioactive labels are known to those skilled in the art and can be a radioisotope such as, for example, ³²P, ³³P, ³⁵S, ³H and ¹²⁵I. Suitable enzyme labels can be, for example, colorimetric labels and chemiluminescence labels. Suitable colorimetric (chromogenic) labels can be, for example, alkaline phosphatase, horse radish peroxidase, biotin and digoxigenin. Biotin can be detected using, for example, an anti-biotin antibody, or by streptavidin or avidin or a derivative thereof which retains biotin binding activity conjugated to a chromogenic enzyme such as, for example, alkaline phosphatase and horse radish peroxidase. Digoxigenin can be detected using, for example, an anti-digoxigenin antibody conjugated to a chromogenic enzyme such as, for example, alkaline phosphatase and horse radish peroxidase. Chemiluminescence labels can be, for example, alkaline phosphatase, glucose-6-phosphate dehydrogenase, horseradish peroxidase, Renilla luciferase, and xanthine oxidase. A particularly suitable label can be, for example, SYBR® Green (commercially available from Life Technologies). A particularly suitable probe can be, for example, an oligonucleotide labelled with SYBR® Green. Suitable chemical labels can be, for example, periodate and 1-Ethyl-3-[3-dimethylaminopropyl]carbodiimide hydrochloride (EDC).

The methods can further include measuring the individual's mandible size and categorizing the individual's mandible size in a group selected from a measurement more than one standard deviation below (less than) the average mandible measurement obtained from a control group, a measurement more than one standard deviation above (greater than) the average mandible measurement obtained from a control group, and a measurement from one standard deviation below (less than) the average mandible measurement obtained from a control group to a measurement of one standard deviation above (greater than) the average mandible measurement obtained from a control group.

A particularly suitable method for measuring the individual's mandible size is by measurement of an X-ray of the individual's mandible from condylion to gnathion. Another method for measuring the individual's mandible size is by using a cone beam to image the mandible.

As understood by those skilled in the art, although there are variations of normal, values have been established for the normal size of mandibles. Measurements can be made to measure the ramus, corpus, gonial angle and size as a whole. Cephalometric radiographs of a population in Michigan that was separated based on age and gender were used to establish the average size of the craniofacial skeleton (see, Riolo et al., CGS An Atlas of Craniofacial Growth: Cephalometric Standards from the University School Growth Study, The University of Michigan; (1974), 114:106-107). These findings provide an average size and standard deviation for common cephalometric measurements for each gender from age six years to sixteen years. Condylion to gnathion is a measurement of mandibular size, whereas measurement from articulare to gnathion is one of mandibular position since it is measuring the cranial base position to the chin.

### Methods for Diagnosing Large Mandibular Size

In another aspect, the present disclosure is directed to a method for diagnosing large mandibular size in an individual. In one embodiment, the present disclosure is directed to a method for diagnosing an individual as at risk for large mandibular size.

The method includes: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the nucleic acid sequence; and diagnosing large mandibular size in the individual if the expression level of MSX1 is from about 5 times to about 12 times greater than the expression level of MSX1 from a control group; if the expression level of DLX6 is from about 5.5 times to about 9.2 times greater than the expression level of DLX6 from a control group; and if the expression level of END1 is from about 1 time to about 3 times greater than the expression level of EDN1 from a control group. In some embodiments, the control group includes individuals whose mandibles have a z-score calculated to be from −1 to +1 standard deviation from the average. In other embodiments, the expression level of MSX1 is from about 3.5 times to about 8.5 times greater than the expression level of MSX1 from a control group, the expression level of DLX6 is from about 4 times to about 7 times greater than the expression level of DLX6 from a control group, and the expression level of EDN1 is from about 1 time to about 2.5 times greater than the expression level of EDN1 from the control group, wherein the control group includes individuals whose mandibles have a z-score calculated to be from −1 to 0 standard deviation from the average. In other embodiments, the expression level of MSX1 is from about 2 times to about 23 times greater than the expression level of MSX1 from a control group, the expression level of DLX6 is from about 2 times to about 41 times greater than the expression level of DLX6 from a control group, and the expression level of EDN1 is from about 2.5 time to about 8 times greater than the expression level of EDN1 from the control group, wherein the control group includes individuals whose mandibles have a z-score calculated to be from 0 to +1 standard deviation from the average.

In one embodiment, the present disclosure is directed to a method for diagnosing large mandibular size in an individual. The method includes: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing large mandibular size in the individual if the expression level of MSX1 is from about 5 times to about 12 times greater than the expression level of MSX1 from a control group. In this embodiment, the control group includes individuals whose mandibles have a z-score calculated to be from −1 to +1 standard deviation from the average.

In one embodiment, the present disclosure is directed to a method for diagnosing large mandibular size in an individual. The method includes: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing large mandibular size in the individual if the expression level of MSX1 is from about 3.5 times to about 8.5 times greater than the expression level of MSX1 from a control group. In this embodiment, the control group (the “moderate small group”) includes individuals whose mandibles have a z-score calculated to be from −1 to 0 standard deviation from the average.

In one embodiment, the present disclosure is directed to a method for diagnosing large mandibular size in an individual. The method includes: obtaining a sample from the individual; contacting the sample with a probe, wherein the probe forms a complex with Muscle segment homeobox 1 (MSX1); detecting the complex formed by the probe and the MSX1 to determine an expression level of MSX1; and diagnosing large mandibular size in the individual if the expression level of MSX1 is from about 2 times to about 23 times greater than the expression level of MSX1 from a control group. In this embodiment, the control group (the “moderate large group”) includes individuals whose mandibles have a z-score calculated to be from 0 to +1 standard deviation from the average.

In another embodiment, the present disclosure is directed to a method for diagnosing large mandibular size in an individual. The method includes: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing large mandibular size in the individual if the expression level of DLX6 is from about 5.5 times to about 9.2 times greater than the expression level of DLX6 from a control group. In this embodiment, the control group includes individuals whose mandibles have a z-score calculated to be from −1 to +1 standard deviation from the average.

In another embodiment, the present disclosure is directed to a method for diagnosing large mandibular size in an individual. The method includes: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing large mandibular size in the individual if the expression level of DLX6 is from about 4 times to about 7 times greater than the expression level of DLX6 from a control group. In this embodiment, the control group (the “moderate small group”) includes individuals whose mandibles have a z-score calculated to be from −1 to 0 standard deviation from the average.

In another embodiment, the present disclosure is directed to a method for diagnosing large mandibular size in an individual. The method includes: obtaining a sample from the individual; contacting the sample with a probe, wherein the probe forms a complex with Distal-less homeobox 6 (DLX6); detecting the complex formed by the probe and the DLX6 to determine an expression level of DLX6; and diagnosing large mandibular size in the individual if the expression level of DLX6 is from about 2 times to about 41 times greater than the expression level of DLX6 from a control group. In this embodiment, the control group (the “moderate large group”) includes individuals whose mandibles have a z-score calculated to be from 0 to +1 standard deviation from the average.

In another embodiment, the present disclosure is directed to a method for diagnosing large mandibular size in an individual. The method includes: obtaining a sample from the individual; contacting the sample with a probe, wherein the probe forms a complex with Endothelin 1 (EDN1); detecting the complex formed by the probe and the EDN1 to determine an expression level of EDN1; and diagnosing large mandibular size in the individual if the expression level of EDN1 is from about 1 time to about 3 times greater than the expression level of EDN1 from the control group. In this embodiment, the control group includes individuals whose mandibles have a z-score calculated to be from −1 to +1 standard deviation from the average.

In another embodiment, the present disclosure is directed to a method for diagnosing large mandibular size in an individual. The method includes: obtaining a sample from the individual; contacting the sample with a probe, wherein the probe forms a complex with Endothelin 1 (EDN1); detecting the complex formed by the probe and the EDN1 to determine an expression level of EDN1; and diagnosing large mandibular size in the individual if the expression level of EDN1 is from about 1 time to about 2.5 times greater than the expression level of EDN1 from the control group. In this embodiment, the control group (the “moderate small group”) includes individuals whose mandibles have a z-score calculated to be from −1 to 0 standard deviation from the average.

In another embodiment, the present disclosure is directed to a method for diagnosing large mandibular size in an individual. The method includes: obtaining a sample from the individual; contacting the sample with a probe, wherein the probe forms a complex with Endothelin 1 (EDN1); detecting the complex formed by the probe and the EDN1 to determine an expression level of EDN1; and diagnosing large mandibular size in the individual if the expression level of EDN1 is from about 2.5 time to about 8 times greater than the expression level of EDN1 from the control group. In this embodiment, the control group (the “moderate large group”) includes individuals whose mandibles have a z-score calculated to be from 0 to +1 standard deviation from the average.

The level of expression of MSX1, DLX6 and/or EDN1 can be analyzed using methods such as, for example, reverse transcription polymerase chain reaction (RT-PCR), Northern blot analysis, RNase protection assays, microarrays, serial analysis of gene expression (SAGE), Western blot analysis, immunoprecipitation and combinations thereof.

Reverse transcription polymerase chain reaction (RT-PCR) of gene expression can be used to detect MSX1, DLX6 and EDN1 expression levels by creating complementary DNA from RNA obtained from a sample from an individual.

A particularly suitable RT-PCR method for detecting MSX1, DLX6 and EDN1 expression levels is quantitative PCR (RT-qPCR).

For RT-qPCR, the minimum information for publication of quantitative real-time PCR experiments (MIQE) guidelines are used as described herein.

Northern blot analysis can be performed using methods known to those skilled in the art. For example, total RNA can be extracted from the individual's saliva sample. RNA having a poly(A) tail can then be isolated. The isolated RNA can then be separated according to size by electrophoresis and transferred to a membrane by a blotting system such as, for example, vacuum blotting or capillary blotting. The membrane can then be exposed to the probes described herein, wherein the probes form a complex with the target RNA (e.g., MSX1, DLX6 and EDN1). The complex can then be detected using a method such as, for example, radiography, colorimetric, and chemiluminescence.

The MSX1, DLX6 and EDN1 level can be measured by quantifying the signal by densitometry of the X-ray film using a densitometer.

Suitable samples can be, for example, saliva, blood, plasma, serum and a cheek swab. The samples can be further processed using methods to isolate molecules contained in the sample such as, for example, cells, proteins and nucleic acids (e.g., DNA and RNA).

The isolated molecules can also be further processed. For example, cells can be lysed and subjected to methods for isolating proteins and/or nucleic acids contained within the cells. Proteins and nucleic acids contained in the sample and/or in isolated cells can be processed. For example, proteins can be processed for electrophoresis, Western blot analysis, immunoprecipitation and combinations thereof. Nucleic acids can be processed, for example, for polymerase chain reaction, electrophoresis, Northern blot analysis, Southern blot analysis, RNase protection assays, microarrays, serial analysis of gene expression (SAGE) and combinations thereof.

Suitable probes are described herein and can include, for example, nucleic acid probes, antibody probes, and chemical probes. A particularly suitable probe can be, for example, an oligonucleotide probe labelled with SYBR® Green. Particularly suitable nucleic acid probes can be oligonucleotide probes designed using the MSX1 (SEQ ID NO:1), DLX6 (SEQ ID NO:3) and EDN1 (SEQ ID NO:5) nucleotide sequences provided herein. Particularly suitable antibody probes can be designed using the MSX1 (SEQ ID NO:2), DLX6 (SEQ ID NO:4) and EDN1 (SEQ ID NO:6) amino acid sequences provided herein.

In some embodiments, the probe can be a labeled probe. Suitable labels can be, for example, a fluorescent label, an enzyme label, a radioactive label, a chemical label, and combinations thereof. Suitable radioactive labels are known to those skilled in the art and can be a radioisotope such as, for example, ³²P, ³³P, ³⁵S, ³H and ¹²⁵I. Suitable enzyme labels can be, for example, colorimetric labels and chemiluminescence labels. Suitable colorimetric (chromogenic) labels can be, for example, alkaline phosphatase, horse radish peroxidase, biotin and digoxigenin. Biotin can be detected using, for example, an anti-biotin antibody, or by streptavidin or avidin or a derivative thereof which retains biotin binding activity conjugated to a chromogenic enzyme such as, for example, alkaline phosphatase and horse radish peroxidase. Digoxigenin can be detected using, for example, an anti-digoxigenin antibody conjugated to a chromogenic enzyme such as, for example, alkaline phosphatase and horse radish peroxidase. Chemiluminescence labels can be, for example, alkaline phosphatase, glucose-6-phosphate dehydrogenase, horseradish peroxidase, Renilla luciferase, and xanthine oxidase. A particularly suitable label can be, for example, SYBR® Green (commercially available from Life Technologies). A particularly suitable probe can be, for example, an oligonucleotide labelled with SYBR® Green. Suitable chemical labels can be, for example, periodate and 1-Ethyl-3-[3-dimethylaminopropyl]carbodiimide hydrochloride (EDC).

The methods can further include measuring the individual's mandible size and categorizing the individual's mandible size in a group selected from a Co-Gn measurement more than one standard deviation below (less than) the average mandible Co-Gn measurement obtained from a control group, a measurement more than one standard deviation above (greater than) the average mandible Co-Gn measurement obtained from a control group, and a Co-Gn measurement from one standard deviation below (less than) the average mandible Co-Gn measurement obtained from a control group to a Co-Gn measurement of one standard deviation above (greater than) the average mandible Co-Gn measurement obtained from a control group.

A particularly suitable method for measuring the individual's mandible size is by measurement of an X-ray of the individual's mandible from condylion to gnathion. Another method for measuring the individual's mandible size is by imaging an individual's mandible using a cone beam.

As understood by those skilled in the art, although there are variations of normal, values have been established for the normal size of mandibles. Measurements can be made to measure the ramus, corpus, gonial angle and size as a whole. Cephalometric radiographs of a population in Michigan that was separated based on age and gender were used to establish the average size of the craniofacial skeleton (see, Riolo et al., CGS An Atlas of Craniofacial Growth: Cephalometric Standards from the University School Growth Study, The University of Michigan; (1974), 114:106-107). These findings provide an average size and standard deviation for common cephalometric measurements for each gender from age six years to sixteen years. Condylion to gnathion is a measurement of mandibular size, whereas measurement from articulare to gnathion is one of mandibular position since it is measuring the cranial base position to the chin.

### Prognosis of Mandible Size

In another aspect, the present disclosure is directed to a method of prognosing mandibular size in an individual. As used herein, the term “prognosing” and “prognosis” are used according to their ordinary meaning as understood by those skilled in the art to refer to a prediction of how mandible size in an individual will develop. Thus, prognosis of small mandibular size in an individual refers to predicting that the individual is likely to develop a small mandibular size. Similarly, prognosis of large mandibular size in an individual refers to predicting that the individual is likely to develop a large mandibular size.

The method includes: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the target; and providing a prognosis of mandibular size, wherein the prognosis is small mandibular size in the individual if the expression level of MSX1 is from about 6.5 times to about 25 times less than the expression level of MSX1 from a control group; if the expression level of DLX6 is from about 5 times to about 22 times less than the expression level of DLX6 from a control group; and if the expression level of END1 is from about 1.5 times to about 10.5 times less than the expression level of EDN1 from a control group; and wherein the prognosis is large mandibular size in the individual if the expression level of MSX1 is from about 5 times to about 12 times greater than the expression level of MSX1 from a control group; if the expression level of DLX6 is from about 5.5 times to about 9.2 times greater than the expression level of DLX6 from a control group; and if the expression level of END1 is from about 1 time to about 3 times greater than the expression level of EDN1 from a control group.

In another aspect, the present disclosure is directed to a method of prognosing small mandibular size in an individual. The method includes: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the target; and providing a prognosis of small mandibular size in the individual if the expression level of MSX1 is from about 6.5 times to about 25 times less than the expression level of MSX1 from a control group; if the expression level of DLX6 is from about 5 times to about 22 times less than the expression level of DLX6 from a control group; and if the expression level of END1 is from about 1.5 times to about 10.5 times less than the expression level of EDN1 from a control group. In some embodiments, the control group includes individuals whose mandibles have a z-score calculated to be from −1 to +1 standard deviation from the average.

In other embodiments, the expression level of MSX1 is from about 4.5 times to about 17.6 times less than the expression level of MSX1 from a control group, the expression level of DLX6 is from about 3.5 times to about 16.5 times less than the expression level of DLX6 from the control group, and the expression level of EDN1 is from about 1 times to about 8.2 times less than the expression level of EDN1 from the control group, wherein the control group (the “moderate small group”) includes individuals whose mandibles have a z-score calculated to be from −1 to 0 standard deviation from the average.

In other embodiments, the expression level of MSX1 is from about 28 times to about 108 times less than the expression level of MSX1 from a control group, the expression level of DLX6 is from about 22 times to about 100 times less than the expression level of DLX6 from the control group, and the expression level of EDN1 is from about 3.5 times to about 27 times less than the expression level of EDN1 from the control group, wherein the control group (the “moderate large group”) includes individuals whose mandibles have a z-score calculated to be from 0 to +1 standard deviation from the average.

In another aspect, the present disclosure is directed to a method of prognosing large mandibular size in an individual. The method includes: obtaining a sample from the individual; contacting the sample with a probe, wherein the probe forms a complex with a target selected from the group consisting of Muscle segment homeobox 1 (MSX1), Distal-less homeobox 6 (DLX6) and Endothelin 1 (EDN1); detecting the complex formed by the probe and the target to determine an expression level of the nucleic acid sequence; and §. In some embodiments, the control group includes individuals whose mandibles have a z-score calculated to be from −1 to +1 standard deviation from the average.

In other embodiments, the expression level of MSX1 is from about 3.5 times to about 8.5 times greater than the expression level of MSX1 from a control group, the expression level of DLX6 is from about 4 times to about 7 times greater than the expression level of DLX6 from a control group, and the expression level of EDN1 is from about 1 time to about 2.5 times greater than the expression level of EDN1 from the control group (the “moderate small group”), wherein the control group includes individuals whose mandibles have a z-score calculated to be from −1 to 0 standard deviation from the average.

In other embodiments, the expression level of MSX1 is from about 2 times to about 23 times greater than the expression level of MSX1 from a control group, the expression level of DLX6 is from about 2 times to about 41 times greater than the expression level of DLX6 from a control group, and the expression level of EDN1 is from about 2.5 time to about 8 times greater than the expression level of EDN1 from the control group, wherein the control group (the “moderate large group”) includes individuals whose mandibles have a z-score calculated to be from 0 to +1 standard deviation from the average.

The level of expression of MSX1, DLX6 and/or EDN1 can be analyzed using methods such as, for example, reverse transcription polymerase chain reaction (RT-PCR), Northern blot analysis, RNase protection assays, microarrays, serial analysis of gene expression (SAGE), Western blot analysis, immunoprecipitation and combinations thereof.

Reverse transcription polymerase chain reaction (RT-PCR) of gene expression can be used to detect MSX1, DLX6 and EDN1 expression levels by creating complementary DNA from RNA obtained from a sample from an individual.

A particularly suitable RT-PCR method for detecting MSX1, DLX6 and EDN1 expression levels is quantitative PCR (RT-qPCR).

For RT-qPCR, the minimum information for publication of quantitative real-time PCR experiments (MIQE) guidelines are used as described herein.

Northern blot analysis can be performed using methods known to those skilled in the art. For example, total RNA can be extracted from the individual's saliva sample. RNA having a poly(A) tail can then be isolated. The isolated RNA can then be separated according to size by electrophoresis and transferred to a membrane by a blotting system such as, for example, vacuum blotting or capillary blotting. The membrane can then be exposed to the probes described herein, wherein the probes form a complex with the target RNA (e.g., MSX1, DLX6 and EDN1). The complex can then be detected using a method such as, for example, radiography, colorimetric, and chemiluminescence.

The MSX1, DLX6 and EDN1 level can be measured by quantifying the signal by densitometry of the X-ray film using a densitometer.

Suitable samples can be, for example, saliva, blood, plasma, serum and a cheek swab. The samples can be further processed using methods to isolate molecules contained in the sample such as, for example, cells, proteins and nucleic acids (e.g., DNA and RNA).

The isolated molecules can also be further processed. For example, cells can be lysed and subjected to methods for isolating proteins and/or nucleic acids contained within the cells. Proteins and nucleic acids contained in the sample and/or in isolated cells can be processed. For example, proteins can be processed for electrophoresis, Western blot analysis, immunoprecipitation and combinations thereof. Nucleic acids can be processed, for example, for polymerase chain reaction, electrophoresis, Northern blot analysis, Southern blot analysis, RNase protection assays, microarrays, serial analysis of gene expression (SAGE) and combinations thereof.

Suitable probes are described herein and can include, for example, nucleic acid probes, antibody probes, and chemical probes. A particularly suitable probe can be, for example, an oligonucleotide probe labelled with SYBR® Green. Particularly suitable nucleic acid probes can be oligonucleotide probes designed using the MSX1 (SEQ ID NO:1), DLX6 (SEQ ID NO:3) and EDN1 (SEQ ID NO:5) nucleotide sequences provided herein. Particularly suitable antibody probes can be designed using the MSX1 (SEQ ID NO:2), DLX6 (SEQ ID NO:4) and EDN1 (SEQ ID NO:6) amino acid sequences provided herein.

In some embodiments, the probe can be a labeled probe. Suitable labels can be, for example, a fluorescent label, an enzyme label, a radioactive label, a chemical label, and combinations thereof. Suitable radioactive labels are known to those skilled in the art and can be a radioisotope such as, for example, ³²P, ³³P, ³⁵S, ³H and ¹²⁵I. Suitable enzyme labels can be, for example, colorimetric labels and chemiluminescence labels. Suitable colorimetric (chromogenic) labels can be, for example, alkaline phosphatase, horse radish peroxidase, biotin and digoxigenin. Biotin can be detected using, for example, an anti-biotin antibody, or by streptavidin or avidin or a derivative thereof which retains biotin binding activity conjugated to a chromogenic enzyme such as, for example, alkaline phosphatase and horse radish peroxidase. Digoxigenin can be detected using, for example, an anti-digoxigenin antibody conjugated to a chromogenic enzyme such as, for example, alkaline phosphatase and horse radish peroxidase. Chemiluminescence labels can be, for example, alkaline phosphatase, glucose-6-phosphate dehydrogenase, horseradish peroxidase, Renilla luciferase, and xanthine oxidase. A particularly suitable label can be, for example, SYBR® Green (commercially available from Life Technologies). A particularly suitable probe can be, for example, an oligonucleotide labelled with SYBR® Green. Suitable chemical labels can be, for example, periodate and 1-Ethyl-3-[3-dimethylaminopropyl]carbodiimide hydrochloride (EDC).

The methods can further include measuring the individual's mandible size and categorizing the individual's mandible size in a group selected from a Co-Gn measurement more than one standard deviation below (less than) the average mandible Co-Gn measurement obtained from a control group, a measurement more than one standard deviation above (greater than) the average mandible Co-Gn measurement obtained from a control group, and a Co-Gn measurement from one standard deviation below (less than) the average mandible Co-Gn measurement obtained from a control group to a Co-Gn measurement of one standard deviation above (greater than) the average mandible Co-Gn measurement obtained from a control group.

A particularly suitable method for measuring the individual's mandible size is by measurement of an X-ray of the individual's mandible from condylion to gnathion. Another method for measuring the individual's mandible size is by imaging an individual's mandible using a cone beam.

As understood by those skilled in the art, although there are variations of normal, values have been established for the normal size of mandibles. Measurements can be made to measure the ramus, corpus, gonial angle and size as a whole. Cephalometric radiographs of a population in Michigan that was separated based on age and gender were used to establish the average size of the craniofacial skeleton (see, Riolo et al., CGS An Atlas of Craniofacial Growth: Cephalometric Standards from the University School Growth Study, The University of Michigan; (1974), 114:106-107). These findings provide an average size and standard deviation for common cephalometric measurements for each gender from age six years to sixteen years. Condylion to gnathion is a measurement of mandibular size, whereas measurement from articulare to gnathion is one of mandibular position since it is measuring the cranial base position to the chin.

Kits

In another aspect, the present disclosure is directed to kits. In one embodiment, kits include at least one probe for detecting a target selected from Muscle segment homeobox 1 (MSX1), Distal-less homeobox 6 (DLX6) and Endothelin 1 (EDN1).

Suitable probes are described herein and can include, for example, nucleic acid probes, antibody probes, and chemical probes. A particularly suitable probe can be, for example, an oligonucleotide probe labelled with SYBR® Green. Particularly suitable nucleic acid probes can be oligonucleotide probes designed using the MSX1 (SEQ ID NO:1), DLX6 (SEQ ID NO:3) and EDN1 (SEQ ID NO:5) nucleotide sequences provided herein. Particularly suitable antibody probes can be designed using the MSX1 (SEQ ID NO:2), DLX6 (SEQ ID NO:4) and EDN1 (SEQ ID NO:6) amino acid sequences provided herein.

In one embodiment, the kit can further include instructions for diagnosing small mandibular size in an individual. In another embodiment, the kit can further include instructions for diagnosing large mandibular size in an individual. In another embodiment, the kit can further include instructions for diagnosing an individual as at risk for small mandibular size. In another embodiment, the kit can further include instructions for diagnosing an individual as at risk for large mandibular size. The instructions can further include Table 1, as described herein, which summarizes the averages of mandibular size and standard deviations according to gender and age, for categorizing the individual's mandible size in a group selected from a measurement more than one standard deviation below (less than) the average mandible measurement obtained from a control group, a measurement more than one standard deviation above (greater than) the average mandible measurement obtained from a control group, and a measurement from one standard deviation below (less than) the average mandible measurement obtained from a control group to a measurement of one standard deviation above (greater than) the average mandible measurement obtained from a control group.

The kit can also include reagents and buffer solutions for performing the method.

Suitable reagents can be, for example, an enzyme capable of performing a polymerase chain reaction, deoxynucleotide triphosphate, and buffer solutions capable of performing a polymerase chain reaction.

The disclosure will be more fully understood upon consideration of the following non-limiting Examples.

### EXAMPLES

### Example 1

In this Example, the expression of six genes was analyzed by RT-qPCR.

Specifically, fifty-four individuals six years of age or older with available cephalograms and undergoing orthodontic treatment were included. The measurement of condylion to gnathion as illustrated in FIGS. 1-3 was recorded for each individual to determine mandibular size. A standard or z-score was calculated for the mandibular measurement. The z-score calculates in standard deviations how far the measurement is from the average. The individual was then sorted into one of the four groups based on the z-score. Averages and standard deviations for each age group were determined from age six to sixteen using Moyer's standards. Averages and standard deviations for individuals over the age of 16 were determined using Ann Arbor, Mich. growth study standards. The individuals were then sorted into groups according to their mandibular size. Table 1 summarizes the averages of mandibular size and standard deviations according to gender and age.

TABLE 1
Mandibular Size Averages and Standard
Deviations by Gender and Age.

| Male Age | Mean | SD | Female Age | Mean | SD |
|---|---|---|---|---|---|
| 6 | 103 | 4.5 | 6 | 110.5 | 4.1 |
| 7 | 105.3 | 3.6 | 7 | 103.3 | 4.4 |
| 8 | 109.2 | 3.8 | 8 | 106.3 | 4.7 |
| 9 | 111.7 | 3.9 | 9 | 108.3 | 5 |
| 10 | 114.5 | 3.9 | 10 | 111.3 | 4.9 |
| 11 | 117.6 | 4.3 | 11 | 113.4 | 4.7 |
| 12 | 119.7 | 4.5 | 12 | 115.7 | 4.6 |
| 13 | 123.1 | 5.5 | 13 | 117.8 | 4.3 |
| 14 | 126.5 | 5.7 | 14 | 119.9 | 4 |
| 15 | 128.7 | 5 | 15 | 122 | 4.9 |
| 16 | 133.6 | 5.4 | 16 | 123.6 | 4 |
| >16 | 132.3 | 6.8 | >16 | 120.2 | 5.3 |

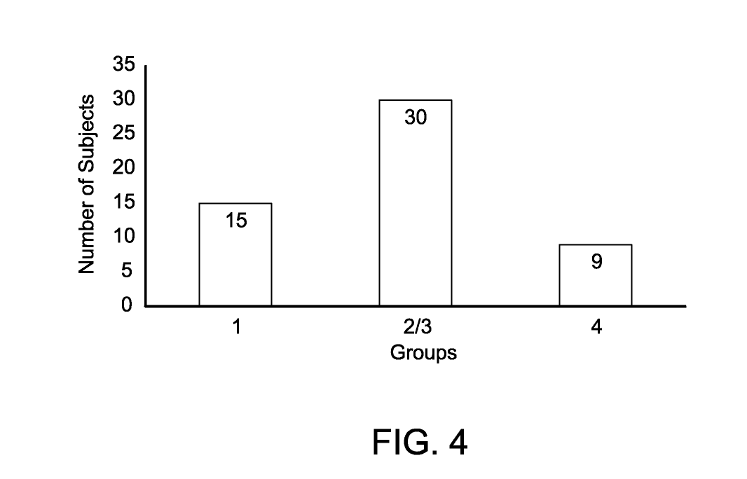

As illustrated in FIG. 4, individuals whose mandibular size was within one standard deviation of the population average were used as the control group (Groups 2/3) to calculate fold regulation for individuals whose mandibular size was greater than or less than one standard deviation from the population average. Group 2/3 was divided into two groups during data analysis due to natural breaks in the data and therefore is referred to Group 2/3 for consistency in labeling. Individuals having a mandible smaller than one standard deviation from the average (Group 1) consisted of 15 individuals. Individuals with a mandible larger than one standard deviation of the average (Group 4) consisted of 9 individuals. Due to the fact that only 56 samples were included in the analysis, effect size was 0.19, alpha was 0.05, and the power was 0.90. Table 2 summarizes each Group.

TABLE 2
Group number assignment and category.

| Group No. | Category |
|---|---|
| 1 | Very small mandible (<−1 = less than 1 SD from the |
| | average mandibular size) |
| 2 | Moderate small mandible (−1 to 0 = within 1 SD of the |
| | average mandibular size) |
| 3 | Moderate large mandible (0 to +1 = within 1 SD of the |
| | average mandibular size) |
| 4 | Very large mandible (>+1 = larger than 1 SD from the |
| | average mandibular size) |

Saliva samples were also collected and handled following minimum information for publication of quantitative real-time PCR experiments (MIQE) guidelines (Bustin et al. Clin. Chem. 2009, 55:611-622). mRNA was extracted from saliva using ORAGENE® RNA purification protocol using the Qiagen RNeasy Micro Kit for volumes up to 1,000 uL (DNA Genotek, Qiagen, Ottawa, ON, Canada). RNA optical density was verified using a NANODROP (ThermoScientific, Wilmington, Del.) and integrity (not degraded) was verified by electrophoresis using a 1.1% agarose gel. The concentration of RNA to be used to prepare cDNA was calculated to provide a total concentration of 400 ng. 400 ng of RNA for each individual was subjected to reverse transcription using iSCRIPT™ Advanced cDNA Synthesis Kit for RT-qPCR

(Bio-Rad, Philadelphia, Pa.). Optical density of the cDNA was verified using a NANODROP (ThermoScientific, Wilmington, Del.).

For qPCR, cDNA was diluted 10-fold in RNA/DNA-free water and subjected to amplification using PrimePCR™ SYBR® Green Assay for Human (Bio-Rad, Philadelphia, Pa.) and SSOADVANCED™ Universal SYBR® Green Supermix (Bio-Rad, Philadelphia, Pa.) for the following genes: muscle segment homeobox 1 (MSX1), distal-less homeobox 5 (DLX5), distal-less homeobox 6 (DLX6), paired-related homeobox gene 1 (PRRX1), endothelin 1 (EDN1) and heart and neural crest derivatives expressed 2 (HAND2). ACTB (beta-actin) and GAPDH (glyceraldehyde 3-phosphate dehydrogenase) were used as normalization controls with three values averaged for each individual. Primers for each gene were obtained from Bio-Rad (Philadelphia, Pa.).

Raw data from qPCR was obtained from the BioRad equipment as Threshold cycle (Ct) values and fold regulation was calculated using the delta-delta-Ct (ddCt) method (see, Schmittgen and Livak, Nat. Protocols 3(6):1101-1108 (2008)). The fold regulation shows how many times up or down the gene is expressed compared to the normalized values obtained from the control group. The fold regulation was calculated only for samples with more than one raw value from qPCR. The raw values for each individual were averaged for each gene. The average expression of the reference genes (ACTB and GAPDH) was calculated for each individual. The average expression of DLX6, EDN1 and MSX1 was calculated for Groups 2 and 3 (as previously described) for each gene to serve as the control group. The average of the individual's reference genes was subtracted from the raw value to give the normalized Ct value with respect to the housekeeping genes. The average value for each individual for DLX6, EDN1, and MSX1 was compared using the average value of Group 2 and 3 for that particular gene. The fold regulation was calculated from these normalized values. The numbers for each group were different for each gene because a valid qPCR reading (at least three independent Ct values) was not obtained for some individuals. Additionally, individuals with a single Ct reading or a Ct value greater than 38 were not included in the analysis. A Ct value greater than 38 was not considered a valid reading because a number of 38 or over 38 could be obtained in a negative control. This means that a sample without expression of a gene could generate a Ct value of 38 or greater than 38. Ct values are larger when there is less gene expression and smaller with more gene expression. Means and standard deviations of Ct values were calculated for the each individual and each gene then averaged. The average Ct and standard deviations as bars above the column for each group is shown in FIG. 8.

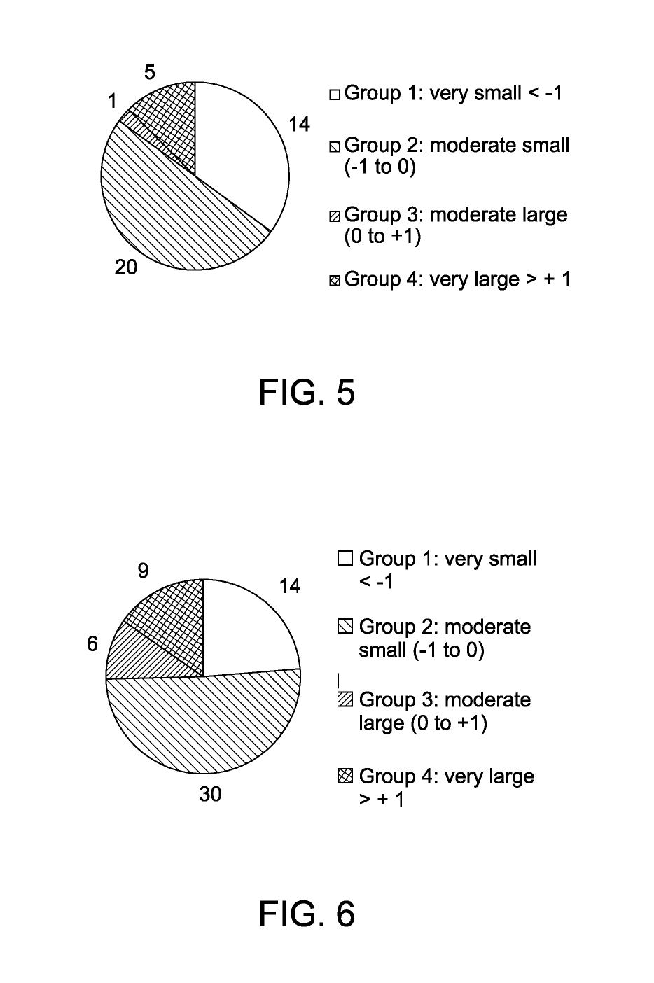

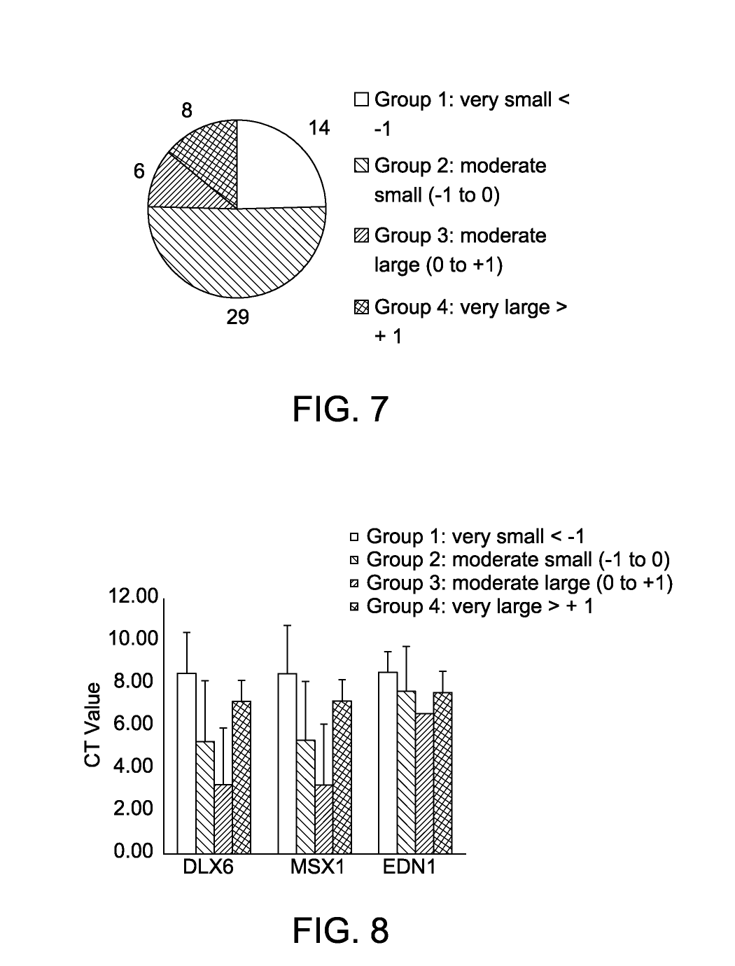

FIGS. 5-7 show the number of individuals for EDN1 (FIG. 3), DLX6 (FIG. 4) and MSX1 (FIG. 5). As illustrated in FIG. 5, 20 Group 2 individuals (i.e., those with moderate small mandible measurements), 14 Group 1 individuals (i.e., those with very small mandible measurements), 5 Group 4 individuals (i.e., those with very large mandible measurements) and 1 Group 3 individual (i.e., those with very small mandible measurements) had detectable levels of EDN1. As illustrated in FIG. 6, 30 Group 2 individuals (i.e., those with moderate small mandible measurements), 14 Group 1 individuals (i.e., those with very small mandible measurements), 9 Group 4 individuals (i.e., those with very large mandible measurements) and 6 Group 3 individual (i.e., those with very small mandible measurements) had detectable levels of DLX6. As illustrated in FIG. 7, 29 Group 2 individuals (i.e., those with moderate small mandible measurements), 14 Group 1 individuals (i.e., those with very small mandible measurements), 8 Group 4 individuals (i.e., those with very large mandible measurements) and 6 Group 3 individual (i.e., those with very small mandible measurements) had detectable levels of MSX1. No samples had a valid reading for the genes DLX5, HAND2 or PRRX1, and thus, may not be expressed in saliva at detectable levels.

The mean of each group was compared to the mean of every other group within the readings for each gene by ANOVA. As shown in FIG. 8, a statistically significant difference was observed for DLX6 and MSX1 between Groups 1 and 2, Groups 1 and 3 and Groups 3 and 4. The statistically significant difference is indicated by an asterisk in the significance column of the post-hoc Tukey HSD test (Table 1).

TABLE 1
Post-Hoc Tukey HSD Analysis.

| | | | | DLX6 | | | MSX1 | | |
|---|---|---|---|---|---|---|---|---|---|
| | | | | | 95% | | | 95% | |
| | | | | | Confidence | | | Confidence | |
| | | | | | Interval | | | Interval | |
| Tukey HSD | | | | | Lower | Upper | | Lower | Upper |
| Group | | Group | | Significance | Bound | Bound | Significance | Bound | Bound |
| 1 | very | 2 | moderate small (−1 to 0) | *0.00 | 1.02 | 5.40 | *0.00 | 0.87 | 5.40 |
| | small <−1 | 3 | moderate large (0 to +1) | *0.00 | 1.94 | 8.54 | *0.00 | 1.86 | 8.64 |
| | | 4 | very large >+1 | 0.62 | −1.56 | 4.22 | 0.69 | −1.80 | 4.37 |
| 2 | moderate | 1 | very small <−1 | *0.00 | −5.40 | −1.02 | *0.00 | −5.40 | −0.87 |
| | small (−1 | 3 | moderate large (0 to +1) | 0.29 | −0.99 | 5.06 | 0.29 | −1.00 | 5.23 |
| | to 0) | 4 | very large >+1 | 0.22 | −4.45 | 0.69 | 0.30 | −4.63 | 0.93 |
| 3 | moderate | 1 | very small <−1 | *0.00 | −8.54 | −1.94 | *0.00 | −8.64 | −1.86 |
| | large (0 | 2 | moderate small (−1 to 0) | 0.29 | −5.06 | 0.99 | 0.29 | −5.23 | 1.00 |
| | to +1) | 4 | very large >+1 | *0.03 | −7.48 | −0.35 | *0.03 | −7.72 | −0.21 |
| 4 | very large | 1 | very small <−1 | 0.62 | −4.22 | 1.56 | 0.69 | −4.37 | 1.80 |
| | >+1 | 2 | moderate small (−1 to 0) | 0.22 | −0.69 | 4.45 | 0.30 | −0.93 | 4.63 |
| | | 3 | moderate large (0 to +1) | *0.03 | 0.35 | 7.48 | *0.03 | 0.21 | 7.72 |

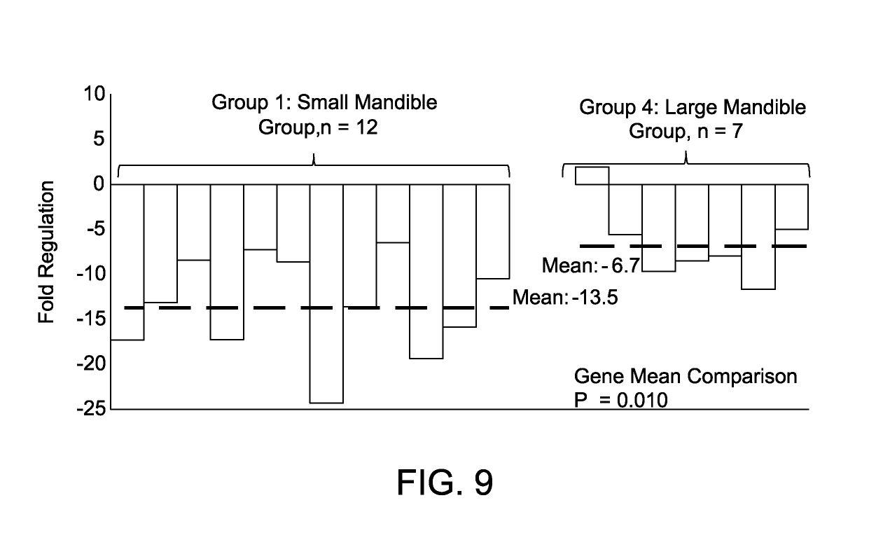

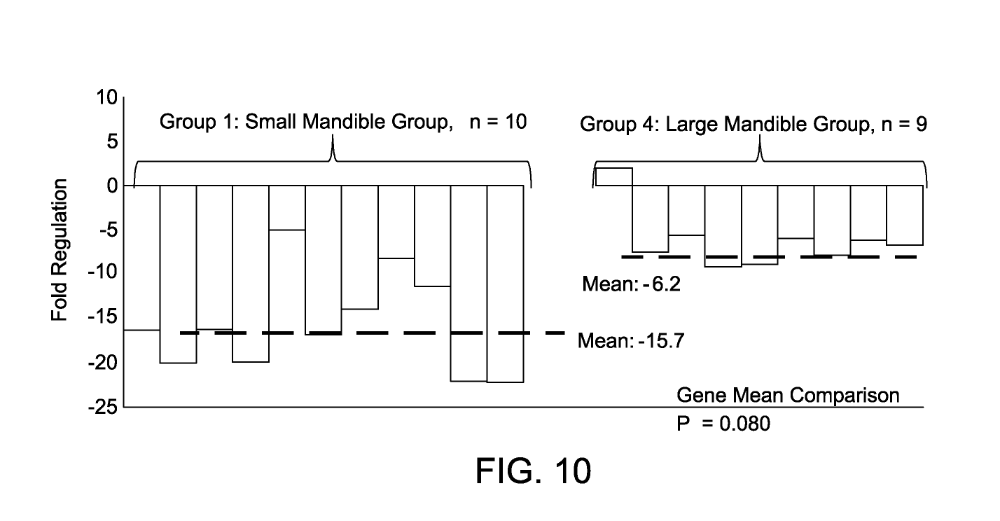

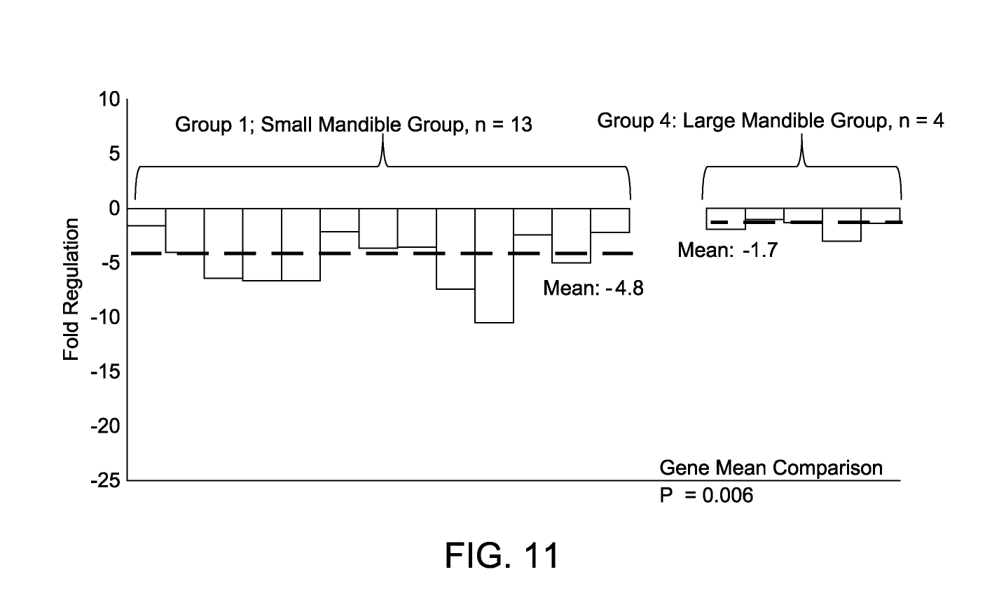

FIGS. 9-11 show the fold regulation in the small (Group 1) and large (Group 4) mandible individuals as compared to the average gene expression for each gene in the control group (having a z-score of −1 to +1). The fold regulation indicates the number of times more or less that a gene is expressed than the gene is expressed in the control group. For example, if the fold regulation is −2.3, then that sample expressed that gene 2.3 times less than the control group expressed that gene. A negative number indicates that the gene was expressed less than the control group, while a positive number indicates more gene expression than the control group. The control group in FIGS. 9-11 was calculated by averaging the expression of Groups 2 and 3. The dotted line through the samples in the graphs is used to indicate the mean for the group.

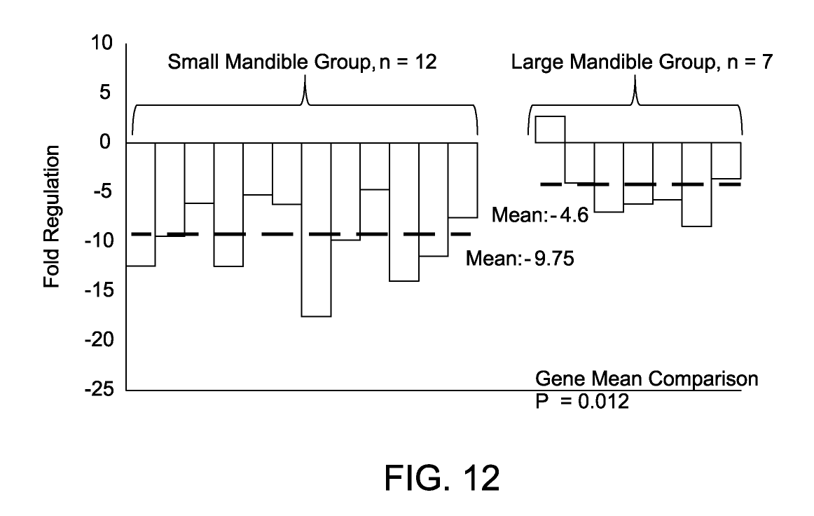

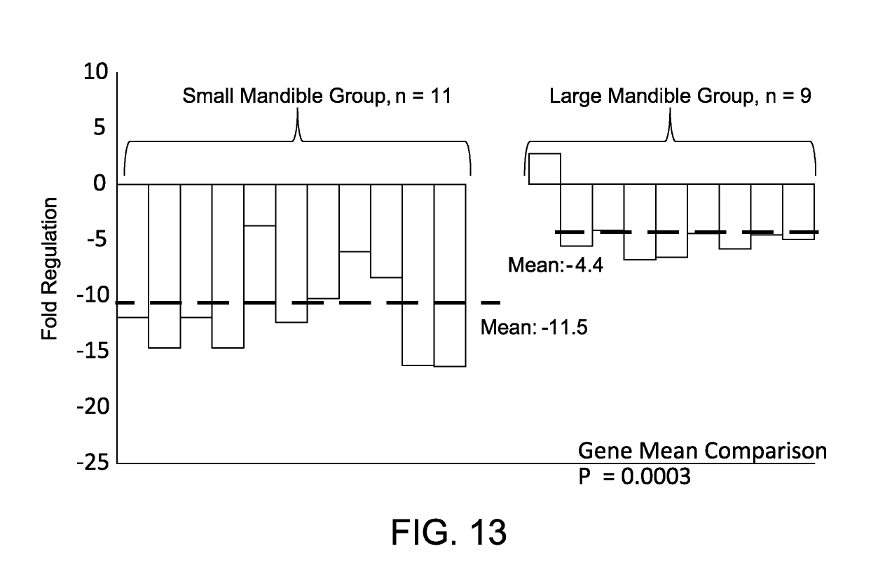

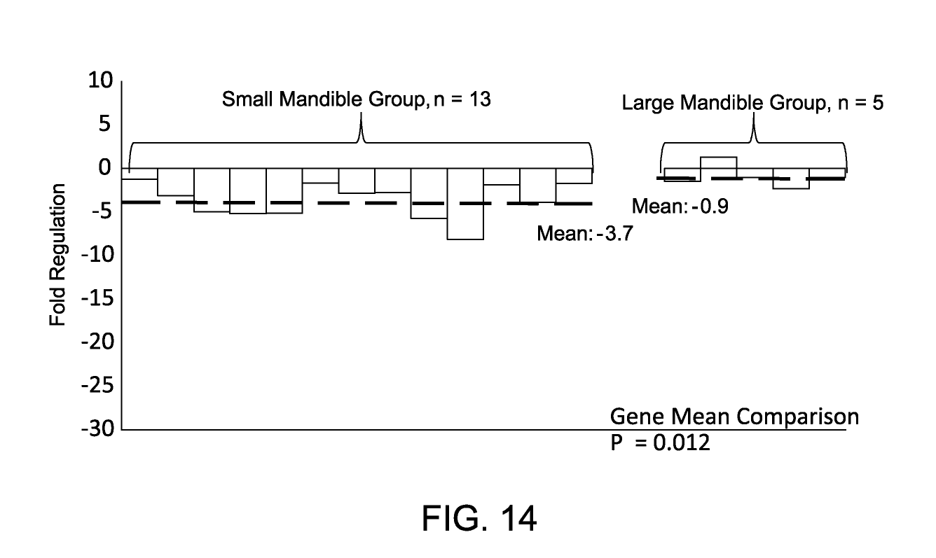

FIGS. 12-14 show the fold regulation in the small (Group 1) and large (Group 4) mandible individuals as compared to the average gene expression for each gene in Group 2 (those with moderate small mandible measurements having a z-score of −1 to 0). Using only Group 2 as the control for comparison to Groups 1 and 4, the fold regulation of expression of MLX1 (FIG. 12), DLX6 (FIG. 13) and EDN1 (FIG. 14) was statistically significant.

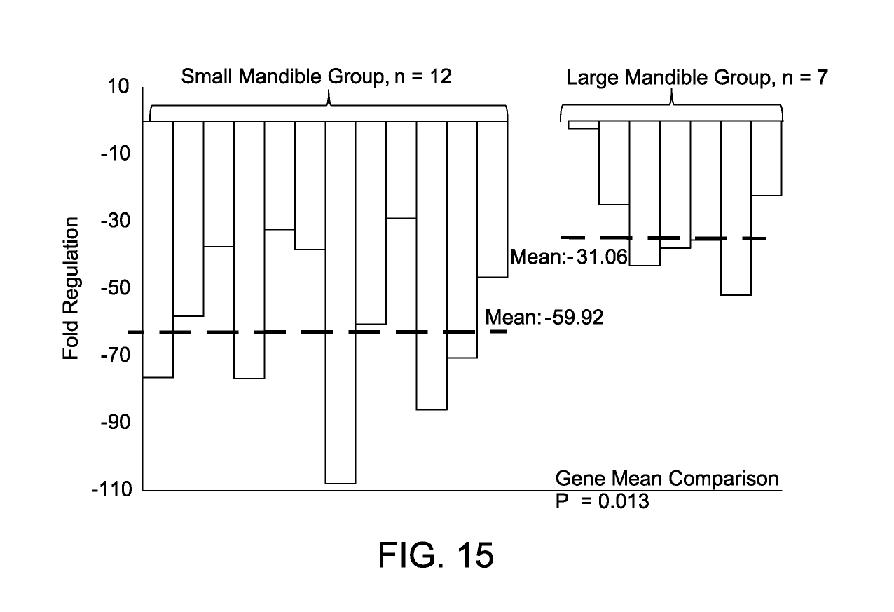

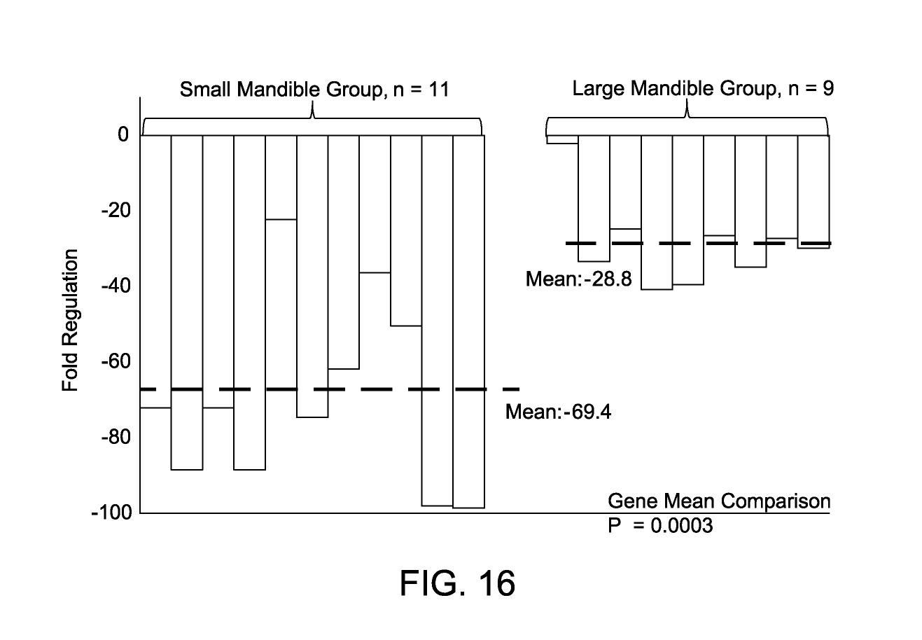

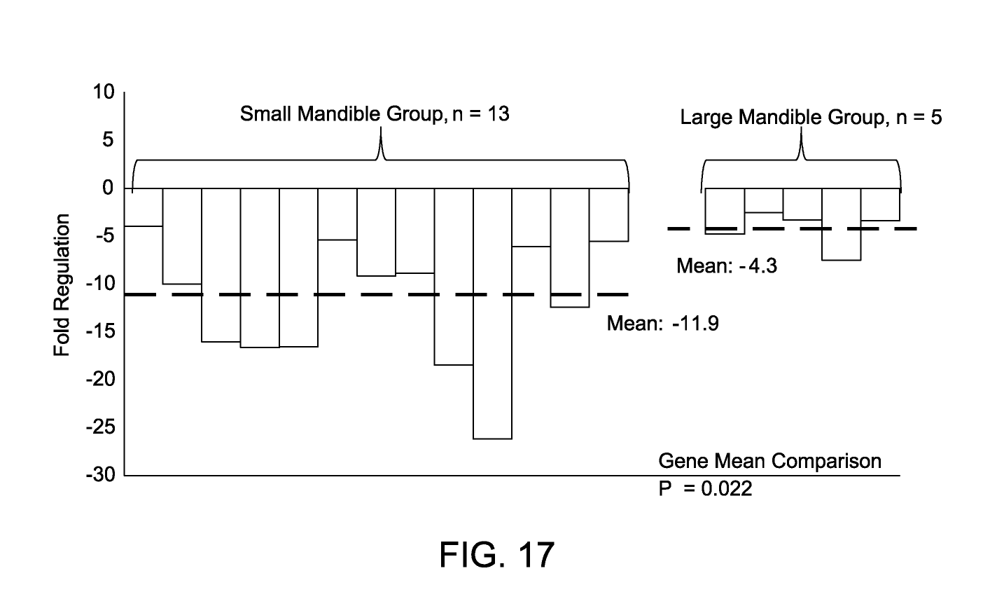

FIGS. 15-17 show the fold regulation in the small (Group 1) and large (Group 4) mandible individuals as compared to the average gene expression for each gene in Group 3 (those with moderate large mandible measurements having a z-score of 0 to +1). Using only Group 3 as the control for comparison to Groups 1 and 4, the fold regulation of expression of MLX1 (FIG. 15), DLX6 (FIG. 16) and EDN1 (FIG. 17) was statistically significant.

These results demonstrated that individuals with a very small mandible had a less MSX1, DLX6 and EDN1 gene expression as compared to all Groups (Groups 2-4). Additionally, individuals with a very large mandible also had a less MSX1, DLX6 and EDN1 gene expression as compared to Groups 2 and 3.

HAND2, DLX5 and PRRX1 did not show any expression in the saliva samples. The Ct values for individuals with a mandible smaller than one standard deviation from the population mean had the least amount of expression of DLX6 and MSX1. The Ct values for individuals with a mandible larger than one standard deviation from the population mean had less expression of DLX6 and MSX1 than individuals whose mandibular size fell within one standard deviation of the population mean. Ct values for EDN1 did not show any statistically significant differences between any of the groups.

DLX6 and MSX1 had the least expression in individuals with mandibular size less than one standard deviation from the population mean. DLX6 and MSX1 have less expression than the control group for individuals with mandibular size more than one standard deviation, but it is not as low as individuals more than one standard deviation from the population mean.

In view of the above, it will be seen that the several advantages of the disclosure are achieved and other advantageous results attained. As various changes could be made in the above methods and systems without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method for diagnosing small mandibular size in an individual, the method comprising:
obtaining a sample from the individual;
contacting the sample with at least one probe, wherein the at least one probe forms a complex with a target selected from the group consisting of Muscle segment homeobox 1 (MSX1), Distal-less homeobox 6 (DLX6) and Endothelin 1 (EDN1);
detecting the complex to determine an expression level of the target in the sample; and
diagnosing small mandibular size in the individual if the expression level of MSX1 in the sample is from about 6.5 times to about 25 times less than the expression level of MSX1 in a sample obtained from a control group; and; if the expression level of DLX6 in the sample is from about 5 times to about 22 times less than the expression level of DLX6 in a sample obtained from the control group; and if the expression level of EDN1 in the sample is from about 1 times to about 10.5 times less than the expression level of EDN1 in a sample obtained from a control group.

2. The method of claim 1, wherein the sample is selected from the group consisting of a saliva sample, a blood sample, a serum sample, a plasma sample and a cheek swab.

3. The method of claim 1, wherein detecting the complex is performed using quantitative polymerase chain reaction.

4. The method of claim 1, wherein the control group consists of individuals having a Condylion-Gnathion measurement within one standard deviation of the average Condylion-Gnathion measurement of the control group.

5. The method of claim 1, further comprising obtaining the individual's Condylion-Gnathion measurement and categorizing the individual's Condylion-Gnathion measurement in a group selected from a Condylion-Gnathion measurement more than one standard deviation less than the average Condylion-Gnathion measurement obtained from a control group, a Condylion-Gnathion measurement more than one standard deviation greater than the average Condylion-Gnathion measurement obtained from a control group, and a Condylion-Gnathion measurement from one standard deviation less than the average Condylion-Gnathion measurement obtained from a control group to a Condylion-Gnathion measurement of one standard deviation greater than the average Condylion-Gnathion measurement obtained from a control group.

6. The method of claim 1, wherein the probe is a nucleic acid probe.

7. The method of claim 6, wherein the nucleic acid probe is an oligonucleotide primer.

8. A method for diagnosing large mandibular size in an individual, the method comprising:
obtaining a sample from the individual;
contacting the sample with at least one probe, wherein the at least one probe forms a complex with a target selected from the group consisting of Muscle segment homeobox 1 (MSX1), Distal-less homeobox 6 (DLX6) and Endothelin 1 (EDN1);
detecting the complex to determine an expression level of the target in the sample; and
diagnosing large mandibular size in the individual if the expression level of MSX1 is from about 5 times to about 12 times less than the expression level of MSX1 from a control group; the expression level of DLX6 is from about 5 times to about 10 times less than the expression level of DLX6 from the control group; and if the expression level of END1 is from about 1 time to about 3 times greater than the expression level of EDN1 from a control group.

9. The method of claim 8, wherein the sample is selected from the group consisting of a saliva sample, a blood sample, a serum sample, a plasma sample and a cheek swab.

10. The method of claim 8, wherein detecting the complex is performed using quantitative polymerase chain reaction.

11. The method of claim 8, wherein the control group consists of individuals having a Condylion-Gnathion size within one standard deviation of the average Condylion-Gnathion size of the control group.

12. The method of claim 8, further comprising measuring the individual's Condylion-Gnathion size and categorizing the individual's Condylion-Gnathion size in a group selected from a Condylion-Gnathion measurement greater than one standard deviation less than the average Condylion-Gnathion measurement obtained from a control group, a Condylion-Gnathion measurement greater than one standard deviation greater than the average Condylion-Gnathion measurement obtained from the control group, and a Condylion-Gnathion measurement from one standard deviation less than the average Condylion-Gnathion measurement obtained from the control group to a Condylion-Gnathion measurement greater than one standard deviation greater than the average Condylion-Gnathion measurement obtained from the control group.

13. The method of claim 8, wherein the probe is a nucleic acid probe.

14. The method of claim 13, wherein the nucleic acid probe is an oligonucleotide primer.

15. A method of prognosing mandibular size in an individual, the method comprising:
obtaining a sample from the individual;
contacting the sample with a probe, wherein the probe forms a complex with a target selected from the group consisting of Muscle segment homeobox 1 (MSX1), Distal-less homeobox 6 (DLX6) and Endothelin 1 (EDN1);
detecting the complex formed by the probe and the target to determine an expression level of the target; and
providing a prognosis of mandibular size,
wherein the prognosis is small mandibular size in the individual if the expression level of MSX1 is from about 6.5 times to about 25 times less than the expression level of MSX1 from a control group; if the expression level of DLX6 is from about 5 times to about 22 times less than the expression level of DLX6 from a control group; and if the expression level of END1 is from about 1.5 times to about 10.5 times less than the expression level of EDN1 from a control group; and
wherein the prognosis is large mandibular size in the individual if the expression level of MSX1 is from about 5 times to about 12 times greater than the expression level of MSX1 from a control group; if the expression level of DLX6 is from about 5.5 times to about 9.2 times greater than the expression level of DLX6 from a control group; and if the expression level of END1 is from about 1 time to about 3 times greater than the expression level of EDN1 from a control group.

16. The method of claim 15, wherein the sample is selected from the group consisting of a saliva sample, a blood sample, a serum sample, a plasma sample and a cheek swab.

17. The method of claim 15, wherein detecting the complex is performed using quantitative polymerase chain reaction.

18. The method of claim 15, wherein the control group consists of individuals having a Condylion-Gnathion size within one standard deviation of the average Condylion-Gnathion size of the control group.

19. The method of claim 15, further comprising measuring the individual's Condylion-Gnathion size and categorizing the individual's Condylion-Gnathion size in a group selected from a Condylion-Gnathion measurement greater than one standard deviation less than the average Condylion-Gnathion measurement obtained from a control group, a Condylion-Gnathion measurement greater than one standard deviation greater than the average Condylion-Gnathion measurement obtained from the control group, and a Condylion-Gnathion measurement from one standard deviation less than the average Condylion-Gnathion measurement obtained from the control group to a Condylion-Gnathion measurement greater than one standard deviation greater than the average Condylion-Gnathion measurement obtained from the control group.

20. The method of claim 15, wherein the probe is a nucleic acid probe.


SEQUENCE LISTING (6 sequences)

SEQ ID NO: 1 - DNA, 4872 bp, Homo sapiens
agggcccggagccggcgagtgctcccgggaactctgcctgcgcggcggcagcgaccggaggccaggcccagcacgccggagctggcctgctggggaggggcgggaggcgcgcgcgggagggtccgcccggccagggccccgggcgctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggagggcccggagccggcgagtgctcccgggaactctgcctgcgcggcggcagcgaccggaggccaggcccagcacgccggagctggcctgctggggaggggcgggaggcgcgcgcgggagggtccgcccggccagggccccgggcgctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccagtgagtagccagaacccaggcgcagagggagggggccgggtgggggccgggtggggtgtgggacccgagggctcctggtggcctccggcgcctgcgtacctgcagccggtgctagggagccgtgggctgcaaggccgggtcttgcgcctccctccactcccacccaggaagaaggttccagacctcctcgccttggcccagagacgctgcgggtgggagttaacggataggacaccgatgtctgggcaccctgtcctcctgcccccaccaaacgacctcaggggtccatgatccctcatctgatcccaaactctgtttcatcggcttcaccccagcggatgaatgtgtgtggtgcggtatcttccctgcacccggagtttcactttctcgcagtaggagctggtgtcccccagcccctcttccctttcaagtacctctttgcctagaggttccgaagctcctacagaattctacctccccatgccctttgagtttgaggcagatagttggtgctttgggcggatggatgattcaggggtggggacattcaggttccagtggagggggcggggcaccaagtcaattaggggaaggcgcccccgctaatcctatgggaagctcccaaacgtctaggactgagccattaaagtggactccaggtgcccaaggcggttcgctccaaggcctcacggccccctggctgctctactcagagaacacgctcggagatatttcaggagcacgggaaattcccaagttttcctcgtttcctccgattattttgctcggcataatagcagccagatttcaatggcgtgatgctgaggaatgatttttatctggggattaaacgtctttgaaaggccagtccctccctaagcctaatggccggagaaggtggccccgctctgggttgtcgccgctgaagggagtgacgtttctctcggcgcccgcccctcgggcggcccggcggaaagctagttgggggccaagcgcttcccggactcccggtggcctccagcagggaagaagcggggtgttaacacgagatttcgtttgactcacatcctggtggtctgaaagtccaaaggatcgttgtgttttctttgttttgttttgttttttctgtttgtttgtggttgttttttagagaggtgtgaaaaaatgcatacttaggcaaaacccgcgtggtgaaacatcttcgatttgaattcactttctgccgggaaagctgctgcataggcaaagtgtcctttccaacgcttagggccttgggccccaagaccccgaagtcaaagcgatcccggctgtgttgggataatttgttccacattttatccgggggcagtccccagcagaccccatccccgacctgcactagtcctgcgctctgatgcttcttcactgtccacccttgaggtttattttgaagccaaaagaaaaagacagctgggcatgttgatgtctgctgactatgccacaggttgaggggagaggcgatctcaacactccccccgcaacaacatcaacacacacacacacacacacacacaaacgtttgagtggggccagagggccctggcgccaggggtgaacgcgatccaacagaggactgagacaatctaaagaaaaagcccattagaataaagcagcccctcgttctccgctccagatgacactttctgtttctaagagggctggccacagtgcaccctccatgatggtctgcgctgctccatctctggtctgcgggaactactcctagaatcccgtaggagcgaagtgttccggggaaagtgtagaatttgatttggattctatgccacaaaactgcctagccccacactgaagcactccgtgggcactgataaatgtttggccaacgcgtaaaactaaatgtgcccttgggctgggcgcagggcctctttctgcatgttcgtcaactgtattaacatccacctttcctctggatggccctgggaggaggcccgccatgaaggccttcctaagccgccgggcagcacaaaggtgatttcacatcttcccagctgtttaggcctaagatgtggacatcgagccttcaacgtgggtatttttctcctggaatcttagtttcttcatttgcaaaaagtagacaggaacttctcccctgcggggttgcaatgggaattggagaaaatatatttcaagtgccttgcgcgatgcccggcaccgaggcacttggcggcactcaatatctggtattgtttggctattattactacttcttgggctgatcatgctccaatgcttctctcttaaccccttgctttttttttctttcggccctcagggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatagagggtcccaggtcgcccacctgtgggccagccgattcctccagccctggtgctgtacccccgacgtgctcccctgctcggcaccgccagccgccttccctttaaccctcacactgctccagtttcacctctttgctccctgagttcactctccgaagtctgatccctgccaaaaagtggctggaagagtcccttagtactcttctagcatttagatctacactctcgagttaaagatggggaaactgagggcagagaggttaacagatttatctaaggtccccagcagaattgacagttgaacagagctagaggccatgtctcctgcatagcttttccctgtcctgacaccaggcaagaaaagcgcagagaaatcggtgtctgacgattttggaaatgagaacaatctcaaaaaaaaaaaaaaaaaaaaaaaaaaaaaaaaaaaagaaaagagaaaaaaaagactagccagccaggaagatgaatcctagcttcttccattggaaaatttaagacaagttcaacaacaaaacatttgctctggggggcagggaaaacacagatgtgttgcaaaggtaggttgaagggacctctctcttaccagtaccagaaacacaattgtaaaattaaaaaaaaaaaaaaactctttctatttaacagtacatttgtgtggctctcaaacatccctttggaagggattgtgtgtactatgtaatatactgtatatttgaaattttattatcatttatattatagctatatttgttaaataaattaattttaagctacaaaaa

SEQ ID NO: 2 - protein, 303 aa, Homo sapiens
MAPAADMTSLPLGVKVEDSAFGKPAGGGAGQAPSAAAATAAAMGADEEGAKPKVSPSLLPFSVEALMADHRKPGAKESALAPSEGVQAAGGSAQPLGVPPGSLGAPDAPSSPRPLGHFSVGGLLKLPEDALVKAESPEKPERTPWMQSPRFSPPPARRLSPPACTLRKHKTNRKPRTPFTTAQLLALERKFRQKQYLSIAERAEFSSSLSLTETQVKIWFQNRRAKAKRLQEAELEKLKMAAKPMLPPAAFGLSFPLGGPAAVAAAAGASLYGASGPFQRAALPVAPVGLYTAHVGYSMYHLT

SEQ ID NO: 3 - DNA, 5063 bp, Homo sapiens
atgatgaccatgactacgatggctgacggcttggaaggccaggactcgtccaaatccgccttcatggagttcgggcagcagcagcagcagcagcagcaacagcagcagcagcagcagcagcaacagcaacagccgccgccgccgccgccgccgccgccgcagccgcactcgcagcagagctccccggccatggcaggcgcgcactaccctctgcactgcctgcactcggcggcggcggcggcagcggccggctcgcaccaccaccaccaccaccagcaccaccaccacggctcgccctacgcgtcgggcggagggaactcctacaaccaccgctcgctcgccgcctacccctacatgagccactcgcagcacagcccttacctccagtcctaccacaacagcagcgcagccgcccagacgcgaggggacgacacaggtgagaggccgctggggcagctcgcttctcccgcctcccgactgccccctaccccgcccgcccgctcacttcctcgacgcccgggcctccgccggccccctcccccaggcggccccgcgcgcccttggccgggccccgtgcgcgcccgctcgcctggcgcctgcctgccggcctctcccagccgggccccttcccgccgccgcggaccctcagcttccgacgcgcagttcgggctgagtcccgggcccggcgcaggcttcgttcgacgagttagtccacactgcgctctccagcctcctgggtctgtgcgcccttttccttgctccttccctccaggctgtaacccacgtttgacccggctggggacatttctgggccggaactggggagcgtgttttgcttcccagcccacgctgggcaggcagtggcctactttgcgtggggcgccccgtgctcctcagtgactcggagggaagctggcctggctttcgctctggttaaactagtaacagtcaggctctttccagtgggctttaaagtgtccctcaccttcatccctgaccgggccccctatctagcttttcgctacagtgttggctgctcctttgttcgtggaggggaagcaaagaacctcgggctgatggttctcagcacccgagggggacaaagttgccgtgagaggcgcagcctgctcgtgggctggtctgagctgttggttgtgttttgtttgaggatttggtcctcctcaggagagtcatttcaaaacatgccgaggatgcttgatgcactgagcttggagctggtgacatcaagtcaactggccgcgtaaacacctgggtggctcaagcacagcttcaggtactgttaagccgggcacaggggccccgctccgcccggacagctgccgccttgctgcgctgcgcaccccagagagcctccggcaccgcctccgctgcggcaaagcgggctgtttgtctgagggcctctggcgctcccttggcccagacgctgcacattgttcgggccagggactgggtcaaggagattacgcagacgctgggagcacacgggcagagtggaccaagagaggtcggggcatcatagggatttttcgaggtaacccttattgggctttggggagactcgttgcatgccacgccaggagcagggagatggcagcagtagcctcccgggtgaccgctcctctgtattatttgcttacagatcaacaaaaaactacagtgattgaaaacggggaaatcaggttcaatggaaaagggaaaaagattcggaagcctcggaccatttattccagcctgcagctccaggctttaaaccatcgctttcagcagacacagtatctggcccttccagagagagccgaactggcagcttccttaggactgacacaaacacaggtaattcccgagaagcccaagtatccctgaaatgctggtaccgcttgcagatcgggcagggagcacatcaggaggaattgttggcctagtcaaccaaggatggaaggagcttaatgacaaatgccttttgctccagttatgcactttcctgggacaacacagtttggaatgaatgacctggtattgaatactggattggatttgcacatcctcccaaccctcggcgaccccaccccagcctagaatccttttgtgaccaaactcataattcaattgagtgttattccagagcagaacaacatgaaagagaacaaacatttaggagaaaaaggtgtacaagccaggaggcagaataaggttcaacttttttaaggatccttttgtaggccctgggaactaataaggttttcaacccattgacacagaaagaaaacttgtgaaatgaactaagaaaaaacaaagtaggcataggttccaatgaggtttagtaaaggctccaaagtgcaatctatttatcctaggagtgtcaaggtgggtttttctgaagatttccctaagcacggtggttaagtcatctgggtgacagactgatgtggtctgagctgcctccctggagcacaggacttgggtgcagtggcagagttggagatcaaagagaaatgttaccaaaattgtaacaggtgaagccacagttaagtacagttactgaagttctcccatcctactaatttgaaagccatagaaggcatttcagatttcacaagaaggagcttaggtgagtatattaggaccctggtcaaaaagccaggctgcaatcacctgcagtttttgttgattaatttacctacatggagtaagtaggcctcatctgatgtaggtaatcttcagcaaggggacctcaggttttatgagactgttgctgcagactgattgtgtgttgacttaagacagttgttccttaagctctttttcaatttagcctgttttctctttccatattgctattcataatatttggggtttgatatgaagggattccggctattcagtgacttgtatgcgtcttgaaaaagtagattcttatggctagggaattttaaaggtaaaggaagctgcaccataaaatttccttcttcaaataaagtagatctgaaatgttggcctagatatgctcccatcccagattcccgattcacgtttaattttcagaagccctttgacttttaattactatactaacaatcatgtgatgatccttgaccacagtttgactaggcaacatacaaaatagggaatgaaggagctgcagtccttaatccctgcaaggttatggggatatatttatgtctggagaaagatgcttcatgtataatctatggcatatgcaatatatgcatagatatattcaaggcatttagttataaatgtacaggctttaattccttcagtcatttgctccttgtacctttatagagataacagcttttgggtatctctatgcatgtacacggagaaaatgactgtgtaaaatagttattttacatagactaaatttacattactaaattttatgtaaaatgacaagactttcagaggaggtgcattcaattgaatggctttgatttgaaagggctgcttttaaaaatcctttaggctttggacctggtcatctcattcagctttttcttatttatactgttgttttaaacctacgctagcacgcaattctagaattacaataactttaggtatccaggcaagatacctagtatgttgtaggaagtgggactgtaagaggctagaaagaggtggaaggaggcggggacttggcttgtaggcgttggcggtggttgttttttgttttttgtttttttgcttttttaatattgcttctaaatccctgagtgacgttagaggcactggtttgatgtttatgggcctaatgattgctgcatttcttgcaggtgaagatatggtttcagaacaaacgctctaagtttaagaaactgctgaagcagggcagtaatcctcatgagagcgaccccctccagggctcggcggccctgtcgccacgctcgccagcgctgcctccagtctgggacgtttctgcctcggccaagggtgtcagtatgccccccaacagctacatgcctggctattctcactggtactcctctccacaccaggacacgatgcagagaccacagatgatgtgagttgcccaagggaacaccctagggaaacgtctgaacaaggaaaagaggatccgggacctgcttgtatctgcgaaaaggagccaaaggagcaggcttaggagagctcataagtgtggcaagaagccgactaggctcattctctctccctctctctctctctccctctcctttctttttacttcttcctttcctccattccttctttctttccttttcctttctacctttcttttctttttgcctttcaccttttttctcatttaccttctctcttgagcaacgtcagtaattgatcttgcatctcagagagagagaaagagcatgtgtgagagagaaactggtttctatgccagcactcctgaaaccccttactgtaaggatattttctcttaccccttgggatccaggctctgagtctcttctctttgggagtatccatcaaaatgactttttttaaaaacagattttcccccaaccagaagaatctgcacaaacttggcagcgtttttacttgtttaatgagtttaagacattacatggtgaaagagaagcattttggactcctgcatttttatttaccattcccagactgacgagaaaaagaaaattcctcacataacagcccttctctaaagaaaaaggaaaaagtggctgtaagattagaacattgctacaaagggaatgctgcatgttttatcaaaatgcaatgaccaggaatgatggttgattaaaaaaaaacaaaacaaaaaccactctttccccaccccacccccccaaaccctgaactggaatcaggaaagacggaggaaacaatcaaaatcaccattctattgctttgacacctttactaggtgaattggtggcattcacaaagctaatagggacgtttatatcaagaaacatttctgtatatattgttgaattttagttgtacatatactttgtatgtttttgtcttctttcatatatggagtaaaagccacaaaacgctga

SEQ ID NO: 4 - protein, 293 aa, Homo sapiens
MMTMTTMADGLEGQDSSKSAFMEFGQQQQQQQQQQQQQQQQQQQPPPPPPPPPQPHSQQSSPAMAGAHYPLHCLHSAAAAAAAGSHHHHHHQHHHHGSPYASGGGNSYNHRSLAAYPYMSHSQHSPYLQSYHNSSAAAQTRGDDTDQQKTTVIENGEIRFNGKGKKIRKPRTIYSSLQLQALNHRFQQTQYLALPERAELAASLGLTQTQVKIWFQNKRSKFKKLLKQGSNPHESDPLQGSAALSPRSPALPPVWDVSASAKGVSMPPNSYMPGYSHWYSSPHQDTMQRPQMM

SEQ ID NO: 5 - DNA, 6899 bp, Homo sapiens
ggagctgtttacccccactctaataggggttcaatataaaaagccggcagagagctgtccaagtcagacgcgcctctgcatctgcgccaggcgaacgggtcctgcgcctcctgcagtcccagctctccaccgccgcgtgcgcctgcagacgctccgctcgctgccttctctcctggcaggcgctgccttttctccccgttaaaagggcacttgggctgaaggatcgctttgagatctgaggaacccgcagcgctttgagggacctgaagctgtttttcttcgttttcctttgggttcagtttgaacgggaggtttttgatccctttttttcagaatggattatttgctcatgattttctctctgctgtttgtggcttgccaaggagctccagaaacaggtaggcacgctcgttgacttgtaagtctcggaattacaagttagtgtgttcttatccaccttcatgcttttcttgcttctatttttccccgttctttttatgactgcagcttagagagcaagtgtctgagaattattgctgaaagctactttaagtcttctagtgtaaaatgtaaaattcctctattgaatacaattaggtgcaattgactataacatgacattaaaataacttatcgttttattattattattccattatgtgtttccttggcttttaaaaaatgagaagagtatggacatatacaatttagtcaaatgtatgtttgtaatatatgtgtttatacaggtacacaggccatataggaacttaaatcttatttaaacactattttaatagtgtgttaacgtgtaaaatatttaagcattccagcttgaagccaaggaattgtatccagtcgttcaagcaatgtatgttcagtaaaatcacctgcagagcaaaagtctgttgactaactaccgcctccccccccccccgccaccaccccccgcaggcggtttctgggtgaagcagatgttttctttaaaatttgtcatcattgactttaggtttcttttggcaggtttttggcacccaaaacagtgtgagctctcttttcagctttattcacctgtgctgggaggggagctaggataattcttggctgccgaaggatttaggcagtgcgtgtgcatctgcccgggtcccccccgtttttagggtcagtgcactttttttgtcttttcgtgaccctgactaaagagaaaggatgtcaagggaatgaaaatcctggaatgtgtctgatcatttgaaatgtacaaaattgggcagataagctgcatggctaaattgttaggaggaagaggcaaggcagtagtggagaagggggaggcagtggatcccacacaagcctgatgcccagggattcggaattcaaaatccccccagcctaccttcagtcccctgacctgcttctcagccccaccttaggtcactggtttctatggagttaccctcctgaattgaatattgaatagttaatttctctctccaatcattttccccacctaattttgaaagatatacatcatctggggtaccctgtgccctacacagcatgtgaagtggatgggtaccccctaaagagagggtcatcctgaatggggaagtggccccaaagctaggaataactgtgatttcttgtctttagtcatgtgccaatgttaagtaagcttcagtggatagtgctgtcctaccaagttccttgtagaagccagccggattttcaacaggcagcattccacagcatttccctgagcctgcttcaagaggggtgggggaagtcccttttcaggtgtttatctcctctgcatttgtgtaatctccctgaaggtggataagccaagggcatgagggggaggcaaaaggtgaactcatgttaaggagggaaaaaaataaagagcccttttttctgtgtttcttgctgatggcaggctgtgtgcttcatctgcttttatctgctctgctagctctgactctactgtgatccagcatgtctctcggcgtttgaggagacatcccccactgacctgctctttctctccccagcagtcttaggcgctgagctcagcgcggtgggtgagaacggcggggagaaacccactcccagtccaccctggcggctccgccggtccaagcgctgctcctgctcgtccctgatggataaagagtgtgtctacttctgccacctggacatcatttgggtcaacactcccgagtaagtctctagagggcattgtaaccctagtcattcattagcgctggctccactggagcccagttttagagtttcttttctagggactctgaaggtagtccttctaacaccatccaagtgcctcagtggggacagtttccctctattcctgaaaataacgacagcttcgttcttagcaaccaaggggagggtcttctgaggccccgtagctcaggctactcatgatgggacaagcaggaggccactgcacgtttcaaatgaggaactttcagtgagagggcctcagggggacactctcacagtggcatctgatggggtttcgggaataattgccgaggtcagatgtgggttagtgcaacctgtgcttctcatgggagggtggagactgagaggcagaagtgatgatatagagggttagaatcacttaattttacttacagaaaaacctaggctcaaagtgttgaagccatttgtgcaggagtgagtttgtagcagagctagaactggagcccggatttcctttgctgctatattttccctttagaaatgcccatttcagaactgaaatagaaatactgtccataggcttctctttcacctacagagaagaaaagcagatttcctccttctgccctggacactagttcatcatctgtcggaagcagtcataaacaagcacacatttactatgcatacaatgtaccgttatgacaaaggaggaccaaaatccaaacaatatcaaaccacaccaaaaaccacaaggagcctaataattactaaggtgatacttccaaagggaggactttatttcttagatgagaatgaaaatggacacattggaaattattggagagccctctggctatgagtccttccacaaccatatggtaccaccgactggcaggagaaatgtgtgaacatgtgcctcctcctcccccaaccactggggtcggtggggtgacggtggcacttttagcagtatcctccgtggtttgagttgaaaataagttttaaaaatcctgtgagtcatggttttgcattgaaacctcttcccactgtgtacccacaaatagttaactaaatagaccattagaaaaggaagaaaatataaagcagatgccaagcagagatgtcctaatttttgacaaaaaagcaatgttgcttgtgtcaagaagaaactgaactttgtgaagagttgaaatggaattccactgaattagaaaaacttgttttctcctgcctggatacatacagtcagggccattgatgcacaggtgttcctggctgttgttacactttaccctctgaaatgatgctcccaagtgctatgtgatgagctccttgtgtgcccagtggaataggtgtgtccatgtgtcattttaaagactattaattacactaatatagtttctttctctctttggataataggcacgttgttccgtatggacttggaagccctaggtccaagagagccttggagaatttacttcccacaaaggcaacagaccgtgaaaatagatgccaatgtgctagccaaaaagacaagaagtgctggaatttttgccaagcaggaaaagaactcaggtgagcagaaacacctttgcttttcaatcagtttaacagcctcctgaactccttcctatcatggtactgccttcctgttttagagagactaacagagacattgaaagtcagggtaaagctgaatataacattgctgaaatgtttttccttgtgtattttaacagggctgaagacattatggagaaagactggaataatcataagaaaggaaaagactgttccaagcttgggaaaaagtgtatttatcagcagttagtgagaggaagaaaaatcagaagaagttcagaggaacacctaagacaaaccaggtaagagggaaggaagaaaaattaggtaagaggttcacaagaacaactagccccagtcagtgatgccagcagcctgttcctccagcccttcttacccgggcaggtgaaagacttagaaaacagtagcagaggagatctatgcatcctatagattaaaaggagcaaaagaatccctcttaaatatttccatgaagctctggaatgcaaaccgatgtcctctgtacttttagcacataccatttcatctacaggtagatttcccaaccaaaatatatccagagatgcctttgtcattgggttatatacagcctttgcctctctgagtcaatgtatttaccactttccctgagaaatcgaaaatcattttggggagcggacatttagaaaaagaatcaaagtgtcatggataatcaaattcttcaataagttgcagttattcagatggccaaaggaaaaataaagtcattagatagggttggtagaatttagaacatgctgtttttcaggtttatggtcttttttttttttttttttttaaatagggaaatgtgtttggtgcagagccaatgtcattccaaaaagctctctcttttcctggtcagtcatgtgctgggacagagaagggatctggattaggcaacatcatagagttgctctgagctgctctttggtgataacccttccaaatcctaaactttttggaattcacaagctcaaaggaggaaacctactctctgatctaccacatgttctgcatttttctatcatggtctatggaaacttctcttagaaatccagtggcaagaagttctatgattaaagtgttctgagctcaggccaggcagtcatgaactacttctgagttatttactactgatttgtggggcagcctcagctatcggtttcttcacacctgcttatgagagtatccatatttatggtcgcaggccagtaatgctccccacgagatcagtttctgaactaacctggaattttttatgggtttttattatgccaactattaaatcaacattacagttcttccctctgtatttctcctgtaaaacattaggcctgcaaaaaaaaaaaatctttttaaaaataattgccataaagtatttgctctgggcctactgtatgcttcttttctttttctctcttttcaactaagtcaccgtcaatttattaagatggccataactattcaaaacctatgctgagttcctcaaggcagggtcacatagtgatgaaggttgggatggggctacggaagaaaccagaacaactctagtttatttaaaacctgtatttactgcccacttccccttagacttgaccatatgacccctcgctcccattctaagcataggggcaggctttatttttacaatggtaatagatatcacttgaggttttatcaaagagttgcggcgggtggtgaaagttcacaaccagattcaggttttgtttgtgccagattctaattttacatgtttcttttgccaaagggtgatttttttaaaataacatttgttttctcttatcttgctttattaggtcggagaccatgagaaacagcgtcaaatcatcttttcatgatcccaagctgaaaggcaagccctccagagagcgttatgtgacccacaaccgagcacattggtgacagaccttcggggcctgtctgaagccatagcctccacggagagccctgtggccgactctgcactctccaccctggctgggatcagagcaggagcatcctctgctggttcctgactggcaaaggaccagcgtcctcgttcaaaacattccaagaaaggttaaggagttcccccaaccatcttcactggcttccatcagtggtaactgctttggtctcttctttcatctggggatgacaatggacctctcagcagaaacacacagtcacattcgaattcgggtggcatcctccggagagagagagaggaaggagattccacacaggggtggagtttctgacgaaggtcctaagggagtgtttgtgtctgactcaggcgcctggcacatttcagggagaaactccaaagtccacacaaagattttctaaggaatgcacaaattgaaaacacactcaaaagacaaacatgcaagtaaagaaaaaaaaaagaaagacttttgtttaaatttgtaaaatgcaaaactgaatgaaactgttactaccataaatcaggatatgtttcatgaatatgagtctacctcacctatattgcactctggcagaagtatttcccacatttaattattgcctccccaaactcttcccacccctgctgccccttcctccatcccccatactaaatcctagcctcgtagaagtctggtctaatgtgtcagcagtagatataatattttcatggtaatctactagctctgatccataagaaaaaaaagatcattaaatcaggagattccctgtccttgatttttggagacacaatggtatagggttgtttatgaaatatattgaaaagtaagtgtttgttacgctttaaagcagtaaaattattttcctttatataaccggctaatgaaagaggttggattgaattttgatgtacttatttttttatagatatttatattcaaacaatttattccttatatttaccatgttaaatatctgtttgggcaggccatattggtctatgtatttttaaaatatgtatttctaaatgaaattgagaacatgctttgttttgcctgtcaaggtaatgactttagaaaataaatatttttttccttactgta

SEQ ID NO: 6 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW